(19) United States
(12) Patent Application Publication
(10) Pub. No.: US 2017/0216508 A1
(43) Pub. Date: Aug. 3, 2017

(54) COPLANAR WIRELESS ENERGY TRANSFER

(71) Applicant: Leviticus Cardio Ltd., Petach Tikva (IL)

(72) Inventors: Michael Zilbershlag, Givat Shmuel (IL); Neri Naveh, Kiryat Ono (IL)

(21) Appl. No.: 15/481,199

(22) Filed: Apr. 6, 2017

Related U.S. Application Data
(63) Continuation-in-part of application No. 14/041,698, filed on Sep. 30, 2013, now Pat. No. 9,642,958.
Application No. 14/041,698 is a continuation-in-part of application No. 13/588,524, filed on Aug. 17, 2012, now Pat. No. 9,343,224.
(60) Provisional application No. 61/708,333, filed on Oct. 1, 2012.
(60) Provisional application No. 61/540,140, filed on Sep. 28, 2011.
(60) Provisional application No. 61/525,272, filed on Aug. 19, 2011.

(51) Int. Cl. A61M 1/12; H02J 50/12; H01F 38/14

(52) U.S. Cl. CPC A61M 1/127; H01F 38/14; H02J 50/12; A61M 1/122

(57) ABSTRACT

An external transmitter inductive coil can be provided in, on, or with a belt designed to be placed externally around a part of a body of a patient. An implantable device (such as a VAD or other medical device) that is implanted within the patient's body has associated with a receiver inductive coil that gets implanted within that part of the patient's body along with the device. The externally-located transmitter inductive coil inductively transfers electromagnetic power into that part of the body and thus to the receiver inductive coil. The implanted receiver inductive coil thus wirelessly receives the inductively-transferred electromagnetic power, and operates the implant.

### RELATED APPLICATIONS

This application is a continuation-in-part of U.S. Nonprovisional application Ser. No. 14/041,698, filed Sep. 30, 2013. Application Ser. No. 14/041,698 claims the benefit of and priority to U.S. Provisional Application Ser. No. 61/708,333, filed Oct. 1, 2012, and is a continuation-in-part of U.S. Nonprovisional application Ser. No. 13/588,524, filed Aug. 17, 2012, which claims the benefit of and priority to U.S. Provisional Application Ser. No. 61/540,140, filed Sep. 28, 2011, and 61/525,272, filed Aug. 19, 2011. The entirety of each of the above-referenced applications is incorporated herein by reference.

### TECHNICAL FIELD

The present invention generally relates to wireless energy transfer into the body of a patient to power wirelessly a device implanted within the body.

### BACKGROUND

Congestive heart failure results from the inability of the heart to pump blood throughout the body at its normal pace, causing blood to flow a slower rate with increased pressure. As a result, the heart is unable to meet the oxygen and nutrient demands of an individual's vital organs. Heart failure may be caused by cardiomyopathy, heart valves damage, coronary heart disease, hypertension, and, in some cases, diabetes. Worldwide, more than a million patients currently suffer from congestive heart failure. In the United States alone, thousands of patients with congestive heart failure are candidates for heart transplantation or an electro-mechanical heart implant, such as a ventricular assist device.

Ventricular assist devices (VAD) are implantable electro-mechanical pumps that are used to partially or completely replace the function of a failing heart. Ventricular assist devices do not replace the heart entirely, but rather assist the right (RVAD) or left (LVAD) ventricle in their ability to pump blood. The choice of the device depends on the underlying heart disease and the pulmonary arterial resistances, which determines the load on the right ventricle. LVADs are more common, as RVADS are typically only necessary when pulmonary arterial resistance is very high.

VADs require a power source to operate the pump. Typically, the VAD is connected to an external power source by a transcutaneous drive line. The exit site of the drive line from the abdomen provides a portal of entry for pathogens, making VAD recipients highly vulnerable to device-related infections. However infectious complications are not limited to VAD systems, as infections are common in many medical devices that use transcutaneous power line.

As an alternative to the transcutaneous power lines, wireless power transfer systems were developed to deliver power to implanted medical devices, including VADs. The traditional approach is TET (transcutaneous energy transfer), in which the energy source is directed toward the energy harvesting device with the goal to minimize RF exposure of the patient. In one commercial embodiment, the receiver coils are located under the patient's skin and the transmitter above the skin. Such TET systems are very sensitive to misalignment and movement of the implanted coil. Additionally, the coil implanted in a separate surgical procedure. Another shortcoming of the current TET solution is that the electromagnetic field density is so high that it can cause heating of the skin and even burns. That is, when the receiver is receiving energy, regular resistance losses within the coil can cause heating to the same volume of tissue receiving the electromagnetic radiation and add heating to it. When the transmitter attached to the receiver is transmitting energy, regular resistance losses within the transmitter coil can cause heating that adds to the receiver regular resistance losses heating and to the receiving electromagnetic radiation heating. The accumulated heat can become a complex issue. TET systems have also suffered setbacks due to complexity and lack of efficiency.

### SUMMARY

The invention concerns coplanar wireless energy transfer (CET) systems. CET systems of the invention typically include an external transmitter coil and an implantable receiver coil coupled to an implantable medical device. The external transmitter inductive coil can be provided in, on, or with a belt designed to be placed externally around a part of a body of a patient. The external transmitter inductive coil is in communication with the internal receiver coil, as implanted, to provide wireless energy transfer to an implant device associated with the internal receiver coil. The two coils are disposed in a coplanar manner and allow for wireless energy transfer from the transmitter inductive coil to the receiver inductive coil, thereby avoiding the need for wires running inside the body from the implanted device to the under skin implant receiver as in TET system. This invention provides new approaches for medical implant wireless power transfer, which will increase the safety and efficiency, and in parallel reduce the cumbersomeness of traditional TET use by simplify the surgery and placement process.

The transmitter inductive coil can be one, two, or more turns of an electrically-conductive material such as a metal wire. The patient can be a human or an animal, and the part of the body can be the arm, leg, head, or torso of the patient. An implantable device (such as a VAD or other medical device) that is implanted within that part of the patient's body has associated with it (for example, electrically coupled to it) a receiver inductive coil that gets implanted within that part of the patient's body along with the device. The externally-located transmitter inductive coil surrounds at least a portion of the implanted receiver inductive coil and inductively transfers electromagnetic power into that part of the body and thus to the receiver inductive coil. The implanted receiver inductive coil thus wirelessly receives the inductively-transferred electromagnetic power from the external transmitter coil, and the implanted receiver inductive coil provides that received power to the implanted implantable device to allow that device to operate. If the device is a VAD, then the power can be used to operate the pumping action of the VAD. In some embodiments, the receiver includes multiple redundant coils, which allow the device to continue drawing power in the event that one coil becomes disconnected.

Methods and designs for optimizing CET systems and examples of using CET for VAD or LVAD powering are also presented. In certain aspects, the invention provides methods for automatically detecting a target or resonance frequency for optimal transfer of energy from the transmitter inductive coil to the receiver inductive coil. In certain embodiments, the transmitter inductive coil is operably associated with circuitry configured to search a range of frequencies and detect the resonance frequency or a target frequency within that range. The ability to hone in on the resonance or target frequency allows one to maximize the operation efficiency of a CET system. In one embodiment, a frequency range used for wireless energy transfer into the human body is 60 KHz to 1 MHz. In preferred embodiments, the frequency used ranges from 80 KHz to 300 KHz. In further embodiments, the frequency used ranges from 90 KHz to 115 KHz. Additionally, the invention maximizes the design and geometry of the transmitter inductive coil and/or the receiver inductive coil in order to reduce energy loss within a CET system. This includes designing the transmitter inductive coil and/or receiver inductive coil in order to avoid or minimize undesirable skin effect and proximal effect. Skin effect is the undesirable increase of wire resistance due to crowding of alternating current near the surface of the wire. Proximity effect losses occur when eddy currents are induced in one winding layer by currents in adjacent layers. Reduction of losses may be accomplished, according to certain embodiments, by using wires that are specifically designed to carry alternating currents to form the coils, such as Litz wires. The structure of those wires can also be chosen to reduce loss. In some embodiments, a wire of the transmitter inductive coil, the receiver inductive coil, or both is formed from 100 to 600 strands with a gauge ranging from 36 AWG to 48 AWG. In one embodiment, the receiver inductive coil includes 130 to 300 strands with a gauge ranging from 36 AWG to 38 AWG. In another embodiment, a wire of the transmitter inductive coil includes 175 to 400 strands with a gauge ranging from 36 AWG to 38 AWG. In addition, the number of turns of a wire that forms the coil is also provided to maximize efficiency. In one embodiment, the transmitter inductive coil includes 6 to 35 turns of a wire. In some embodiments, the receiver inductive coil includes 5 to 50 turns of a wire with a pitch of at least 0.05 inches between each turn. In other embodiments, the pitch is of at least 0.1 inches. The turns of either coil may be arranged in one or more layers, typically one, two, or three layers. Other design considerations for avoiding loss involve the ratio between the height and radius of the transmitter inductive coil. It has been found that a ratio of the height to the radius of about 0.4 to about 1.5 provides enhanced performance. In some embodiments, the receiver coil may include multiple redundant coils, each of which is configured to operate independently of the others. One may serve as a backup to another, or the redundant coils can take turns powering the implanted device. Redundant coils act as a safety mechanism to allow the device to continue operating even if one coil fails.

Circuits associated with the transmitter inductive coil may be designed to minimize loss and maximize efficiency of coplanar wireless energy transfer. One circuit feature is a capacitor (associated with transmitter or receiver coil) that includes an equivalent series resistance that is less than a resistance of the coil. Another feature includes associating the transmitter inductive coil with a half-bridge pulse generator. Half-bridge pulse generators, despite being square-generators, are advantageously able to generate a sinusoidal wave. In further embodiments, the receiver inductive coil is associated with an AC-to-DC rectification circuit that includes a single diode in a closed cycle. This configuration saves space within the circuitry without resulting in loss.

Placement of the transmitter inductive coil with respect to the receiver inductive coil also affects efficiency of the system. In order to provide better efficiency and less loss during energy transfer, the receiver inductive coil and transmitter inductive coil, in one embodiment, are placed such that a center of the receiver inductive coil is no more than 7 cm from a center of the transmitter inductive coil.

### BRIEF DESCRIPTION OF THE DRAWINGS

In the drawing(s), like reference characters generally refer to the same or similar parts throughout the different views. The drawings are intended to illustrate the details of one or more embodiments according to the invention and/or the principles of the invention.

FIG. 1 is a schematic overview of coplanar wireless energy transfer according to certain embodiments.

FIG. 2 depicts an external belt 102 surrounding an implantable receiver coil 101; the belt can be opened and closed using a buckle 103.

FIG. 3 illustrates an external belt (such as the belt in FIG. 1) worn by an individual.

FIG. 4 shows an implantable circuit with a DC-to-DC converter coupled to the implantable receiver resonance structure 202. The load R₁ can be a VAD or any other power consuming implantable device.

FIG. 5 is a graph showing a relationship between output power and load resistance.

FIG. 6 shows an alternative circuit coupled to the implantable receiver coil. The circuit has the same resonance structure 202 but uses the inductiveness of the implant VAD 402 and controller 401 as a DC to DC.

FIG. 7 is a circuit that can be used to lock the external transmitter resonance frequency to the implanted receiver resonance frequency.

FIG. 8 is another circuit that can be used to lock the external transmitter resonance frequency to the implanted receiver resonance frequency.

FIG. 9 shows ring coil 701 implanted in the bottom of the pericardium sack 702.

FIG. 10 shows two ring coil 801 implanted in the bottom of the pulmonary cage.

FIG. 11 shows stent base ring coil 901 located in the descending aorta 902.

FIG. 12 shows a model circuit for calculating the efficiency of energy transmission.

FIG. 13 shows a schematic of a transmitter circuit.

FIG. 14 shows a schematic of a parallel-loaded receiver circuit.

FIG. 15 shows a schematic whereby a simple single-phase rectifying circuit is added to a receiver.

FIG. 16 is a flow chart of coarse and fine resonance frequency detection.

FIG. 17 is a flow chart of coarse and fine frequency base power control.

FIG. 18 shows a configurable capacitor for use with a circuit for locking the external transmitter resonance.

FIG. 19 exemplifies frequencies that can cause tissue heating.

FIG. 20 depicts dynamic band of frequency used to search for resonance or target frequency.

FIG. 21 illustrates the effect that the height of a transmitter coil has on robustness of power transfer if the proximity effects are ignored.

FIG. 22 illustrates the effect that the height of a transmitter coil has when the proximity effects are considered.

FIG. 23 depicts a preferred circuit for the half bridge Pulse generator.

FIG. 24 illustrates rectification with one diode (half wave) and with a diode bridge (full wave).

FIG. 25 illustrates the geometry of the transmitter coil and receiver coil and their relation to each other.

FIG. 26 depicts a transmitter coil having 16 wire turns arranged in two layers (e.g. 8 turns per layer).

FIG. 27 depicts a separated ring coil according to certain embodiments.

FIG. 28 depicts a separated ring coil disposed within a pleural cavity and pericardium.

FIG. 29 depicts an implantable receiver unit.

FIG. 30 depicts an implantable receiver unit with redundant coils.

FIG. 31A depicts a top view of a configuration of redundant coils.

FIG. 31B depicts a cross-section view of the redundant coils of FIG. 31A.

FIG. 32A depicts a top view of a configuration of redundant coils.

FIG. 32B depicts a cross-section view of the redundant coils of FIG. 32A.

FIG. 33A depicts a top view of a configuration of redundant coils.

FIG. 33B depicts a cross-section view of the redundant coils of FIG. 33A.

FIG. 34 depicts a circuit architecture for redundant coils.

### DETAILED DESCRIPTION

The invention relates to a wireless energy transfer system that wirelessly provides energy to an internal implant using an external transmitter inductive coil and an internal receiver inductive coil. The external transmitter inductive coil can be provided in, on, or with a belt designed to be placed externally around a part of a body of a patient. The external transmitter inductive coil is in communication with the internal receiver coil to provide wireless energy transfer to a device implanted within the body.

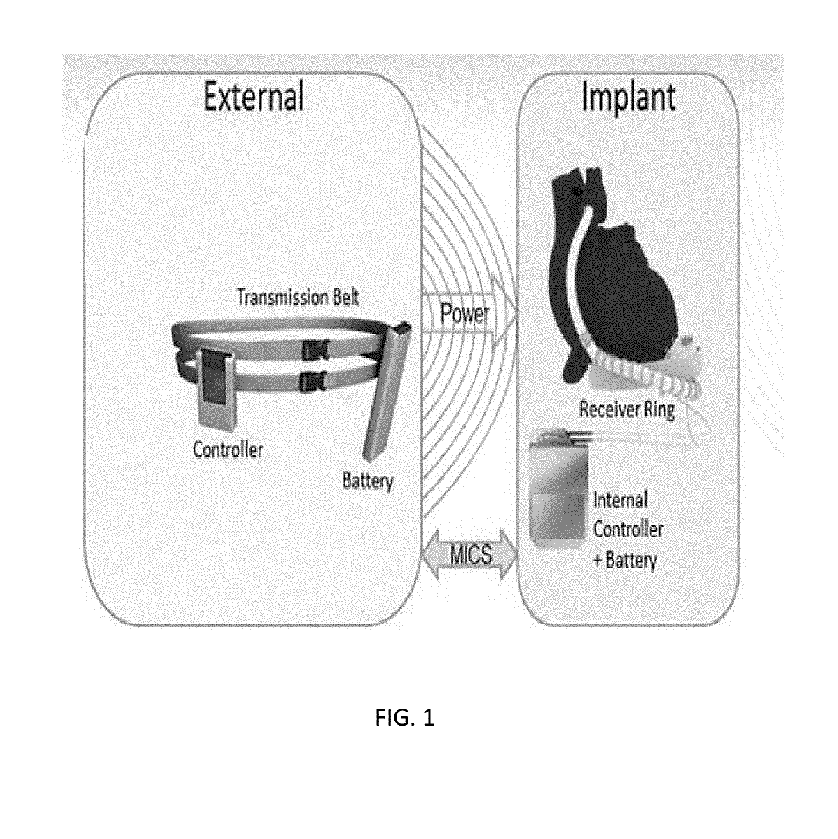

FIG. 1 depicts a schematic overview of wireless energy transfer system of the invention. As shown in FIG. 1, the external components may include a transmission belt (which includes a transmitter inductive coil) a controller, and an external battery. The internal components include an implant (such as a ventricular assist device (VAD)), a receiver inductive coil coupled to the implant, an internal controller, and, optionally, an internal battery. The internal receiver coil is placed within the body (for example, the pericardium sack) and receives power from the external transmission belt. FIG. 3 depicts the external transmission belt disposed an individual's torso, and is ideally placed for transmitting power to an implant in the pericardium sack. The internal controller controls is associated with the receiver, controls power reception circuits, activates the implant electronics, and communicates with the external controller. The internal battery provides back-up power and enables operation of the implant independent of the wireless power transfer. The transmission belt includes a transmitter coil, and transmits power to the internal receiver coil via a magnetic coupling. The external controller can run power transmission algorithms (described hereinafter), communicate with the implant (e.g. through the frequency band of the Medical Implant Communication Service (MICS), which includes frequencies between 402 and 405 MHz), and push power to the belt from the battery. The external battery is able to provide power to the transmission, and allow for generation of the electromagnetic field.

This physical arrangement of the external surrounding transmitter coil and the internally implanted receiver coil (that is disposed at least partially within an imaginary plane cutting through the patient's body and that is formed or defined by the surrounding external transmitter coil) can be referred to as a coplanar arrangement. And the system of the external transmitter coil and the implanted receiver coil thus can be referred to as a coplanar energy transfer (CET) system.

CET is different than a known and common technique referred to as transcutaneous energy transfer (TET). TET only transfers energy through an area of the skin of a patient to a shallowly-implanted receiver just under that area of the skin. CET, in sharp contrast, involves surrounding the implanted receiver coil by placing or wrapping a transmitter coil completely around the part of the patient's body within which the receiver coil is implanted. If the receiver coil is disposed within the brain of the patient, for example, then CET involves disposing the transmitter coil externally around the corresponding part of the head of the patient such that an imaginary plane defined by the surrounding transmitter coil extends through at least a portion of the brain-implanted receiver coil. If the receiver coil is instead implanted within the descending aorta of the patient's vasculature, CET involves disposing the transmitter coil externally around the corresponding part of the patient's chest such that the imaginary plane defined by the surrounding transmitter coil extends through at least a portion of the aorta-implanted receiver coil. These are just two examples of where the transmitter and receiver coils could be located, and other locations are possible such as the arm or the leg of a patient.

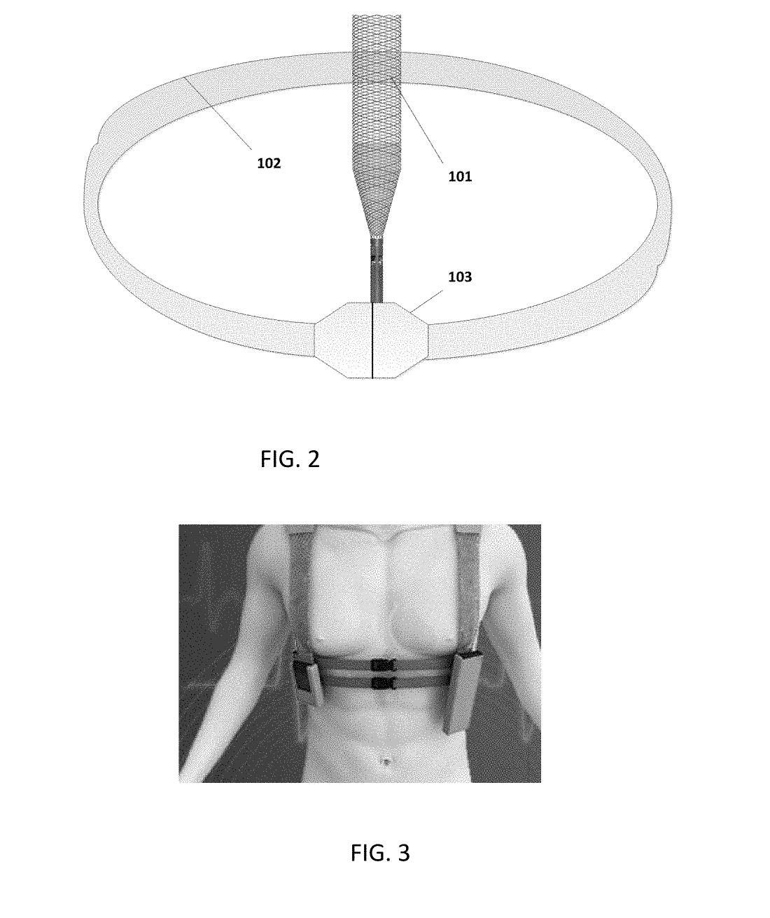

FIG. 2 depicts another view of the coplanar energy transfer system of the invention (also referred to as CET systems). Referring to FIG. 2, a surrounding belt 102 is depicted with a medical stent 101 therewithin. The stent 101 has built into it or incorporated within it a receiver coil of one or more turns of electrically-conductive material such as copper wire, for example. The belt 102 has in or on it, around its entire length, one or more turns of a transmitter coil. Like the receiver coil, the transmitter coil can have one or more turns of electrically-conductive material such as copper wire, for example. Together, the external belt 102 with the transmitter coil and the implantable medical device (such as a stent) with the receiver coil, can be considered a wireless power transfer system. In use, the transmitter coil can be located externally around the chest of a patient or around some other part of the patient's body such as an arm, a leg, a head, or another part of the patient's torso, and the receiver can be implanted within that part of the patient's body, such that electromagnetic power inductively transmitted from the surrounding coil of the belt 102 reaches and is wirelessly received by the patient-implanted receiver coil from all angles and directions.

The stent 101 of FIG. 2 has built into it or incorporated within it the receiver coil, as indicated previously, and in this regard it is noted that the receiver inductive coil can comprise one or more electrically conductive fibers or strands that are among the various fibers or strands that together constitute the stent 101. These fibers or strands that comprise the receiver inductive coil can be electrical wires and can be coated with an electrical insulator. The receiver inductive coil can be built into or incorporated within the stent 101 in a variety of other ways.

In one embodiment, the receiver coil is not built into the device with which it is associated. In this embodiment, the implantable receiver coil is operatively connected (such as by an electrical wire connection) to the implantable device in order to provide wirelessly-received power to the implanted device. The implantable receiver coil can otherwise be physically separate from and not an integral part of the device itself. In another embodiment, the receiver coil is built into the device with which it is associated. In one embodiment, the receiver coil is a stent 101 as shown in FIG. 2 as the device with which the implantable receiver coil is associated.

In addition to VADs, the receiver coil can be associated with a variety of other types of implantable devices, including, for example, a constant glucose meter (CGM), a blood-pressure sensing device, a pulse sensing device, a pacemaker, implantable cardioverter defibrillators (IDC), digital cameras, capsule endoscopies, implanted slow release drug delivery systems (such as implanted insulin pump) a nerve stimulator, or an implanted ultrasound device.

In operation, the CET system generates lower radio-frequency (RF) energy densities than TET systems. Because CET uses a surrounding external belt-like transmitter coil, the RF energy that is inductively transmitted into the patient's body from the transmitter coil is spread out and not concentrated or focused into or onto a particular spot or area of the patient's body. Using CET, the transmitted energy is spread out over the external transmitter coil of the CET, resulting in transmitted field strength and power density levels that are lower than TET systems. Also using a surrounding external belt-like transmitter coil eliminate misalignment problem and reduce dramatically the misplacement problems.

It is noted that a power source must be associated with the external transmitter coil to provide that coil with the power that it will then wirelessly transmit for receipt by the implanted receiver coil. A controller unit also typically will be provided to regulate the operation of the transmitter coil. Like the transmitter coil, both the power source and the controller will be external to the patient. The external source can be an AC current source, and the transmitter coil can be electrically connected to the AC current source. It also is noted that the transmitter coil can be a transmitter—that is, capable of both transmitting and receiving.

### Providing an Optimal Load to the Receiver Resonance Structure:

In one embodiment according to the invention, the device with which the implantable receiver coil is associated is a ventricular assist device (VAD). In this embodiment a DC-to-DC converter is employed to provide an optimal load to the receiver inductive coil. The DC-to-DC converter is designed to automatically adjust to provide a constant or substantially constant selected optimum load to the receiver inductive coil. Typically, the DC-to-DC converter is implanted within the patient's body along with the receiver inductive coil and the VAD.

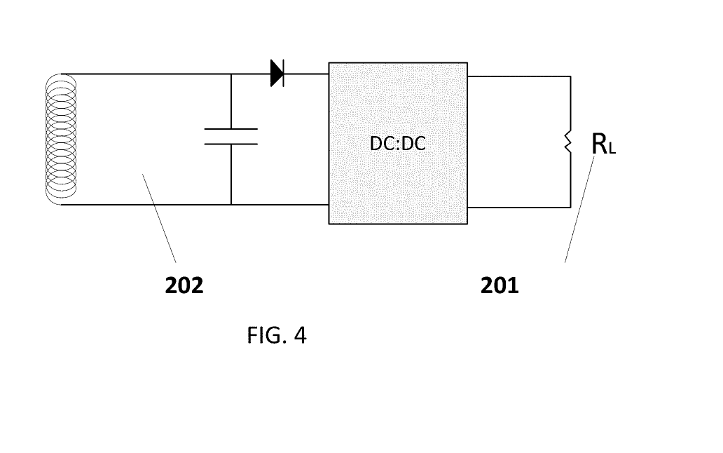

FIG. 4 shows a DC-to-DC converter disposed between the receiver inductive coil and the resonance structure 202 (on the left) and a load R_{L} (on the right). Load 201 may be a VAD or a constant glucose meter, or another implantable device described herein. As shown in FIG. 4, the circuit can also include a half or full-wave rectification (i.e., using a diode or diode bridge). As shown in FIG. 4, resonance structure 202 is formed by the receiver inductive coil and a capacitor. However the external transmitter inductive coil may also be associated with a capacitor to form a transmitter or transmitter resonance structure. A resonance structure of the transmitter can be the same as or different from the receiver.

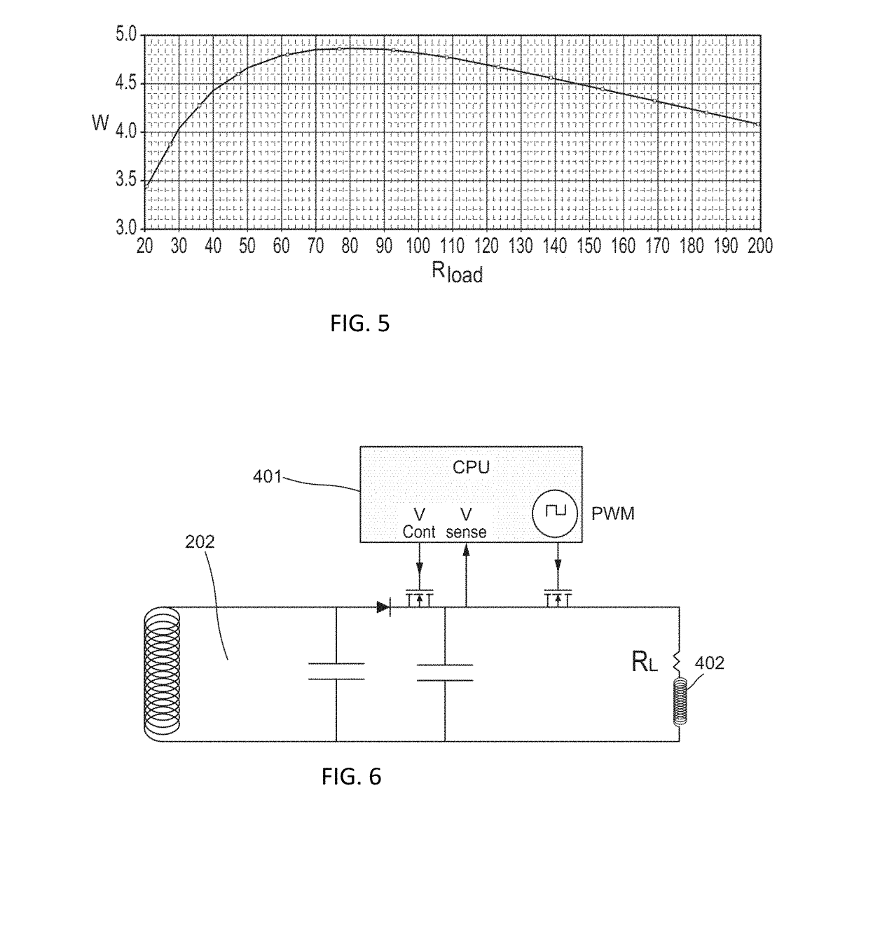

The optimum load can be determined with reference to FIG. 5 which shows a relationship between power harvesting (W) and an R_{load} value for a particular circuit, where R_{load} is the internal resistance of whatever load is associated with the receiver inductive coil. While merely exemplary, the graph of FIG. 5 shows that the best power harvesting for the circuit is 80 Ohms or about 80 Ohms. A load with a resistance lower than 80 Ohms will reduce the voltage on the load and thereby reduce the harvested power, and a load with a resistance higher than 80 Ohms will reduce the current and thereby reduce the harvested power. The shape of the curve in the graph of FIG. 5 is determined by the function (26), provided below. In the case of a VAD, the resistive load represented by the VAD's motor will change as the mechanical load on the motor changes, and the depicted circuit (in FIG. 4) with the DC-to-DC converter is what is used to automatically adjust and provide a substantially constant and optimum load to the receiver inductive coil.

In case of medical implant with high inductive load, like a VAD or implanted slow release drug delivery system that uses a motor, an alternative to the circuit of FIG. 4 is the circuit shown in FIG. 6. The circuit of FIG. 6 can be employed to adjust to an ideal working point of the receiver inductive coil (or, more accurately, of the receiver resonance structure which, as described above, is the combination of the receiver coil and its associated capacitor) when the device with which the implantable receiver coil is associated is a VAD.

An implant with a brushless DC motor, like a VAD, needs adjustable power control to receive exactly the needed mechanical power. As shown in FIG. 5 and by function (26) below and as described above, the best power harvesting is achieved with the optimum R_{load}. In this situation, a high quality motor controller, such as MOTION EN Speed Controller Series SC 1801 F (Faulhaber GmbH & Co. KG, Schonaich, Germany), can be used as a DC-to-DC converter for adjusting the R_{load} to the optimum value using PWM (pulse width modulation). As shown in FIG. 6, a voltage and motor PWM controller 401 gives full control over the working point without any additional measures.

By controlling the voltage and the DC-to-DC rate, the optimum R_{load} can be achieved. The brushless DC motor of the VAD can be simulated with an equivalent resistor and inductor circuit. The speed of the motor is controlled using PWM as the motor input voltage, and the duty cycle is adjusted according to the needed speed. The coils of the VAD's motor flat the current just as is done in DC-to-DC voltage conversion. In this way, the VAD's motor is used as a DC-to-DC converter, and the reflected motor load is dependent on the conversion rate.

Adding a voltage sensor with voltage control adds the capability to select the voltage in the receiver circuit. This gives full control on the reflected load (using the PWM mechanism) and on the used power by controlling the voltage (using the voltage control). For example, a LPC1102 chip can be used for (NXP Semiconductors N.V., Eindhoven, Netherlands) voltage sensing while an internal PWM engine and can control the voltage by using a transistor like SI8409DB (NXP Semiconductors) for closing the inline from the resonance structure 202.

The voltage control can be done in several ways. One example is harvesting control on/off measured, as shown in FIG. 6, in the implanted receiver electronics itself. Another example is transmitting power control in the external transmitter/transmitter primary electronics that closes the loop according to the Vₛₑₙₛₑ in the receiver.

Locking the Receiver and the Transmitter:

Once placed within the body of a patient, the receiver coil shape can be distorted or modified from its at-rest shape and also can move over time to some extent as the patient moves, all depending on the particular location internally within the patient's body where the receiver coil is placed. With changing of its shape, the resonance frequency of the receiver coil changes. It is important for the transmitter resonance structure to be able to automatically find the receiver's resonance and adjust the transmitter's resonance to that found for the receiver and lock to that found resonance. In other words, the transmitter must have the capability to detect the receiver's resonance frequency and then lock to that detected receiver's resonance frequency.

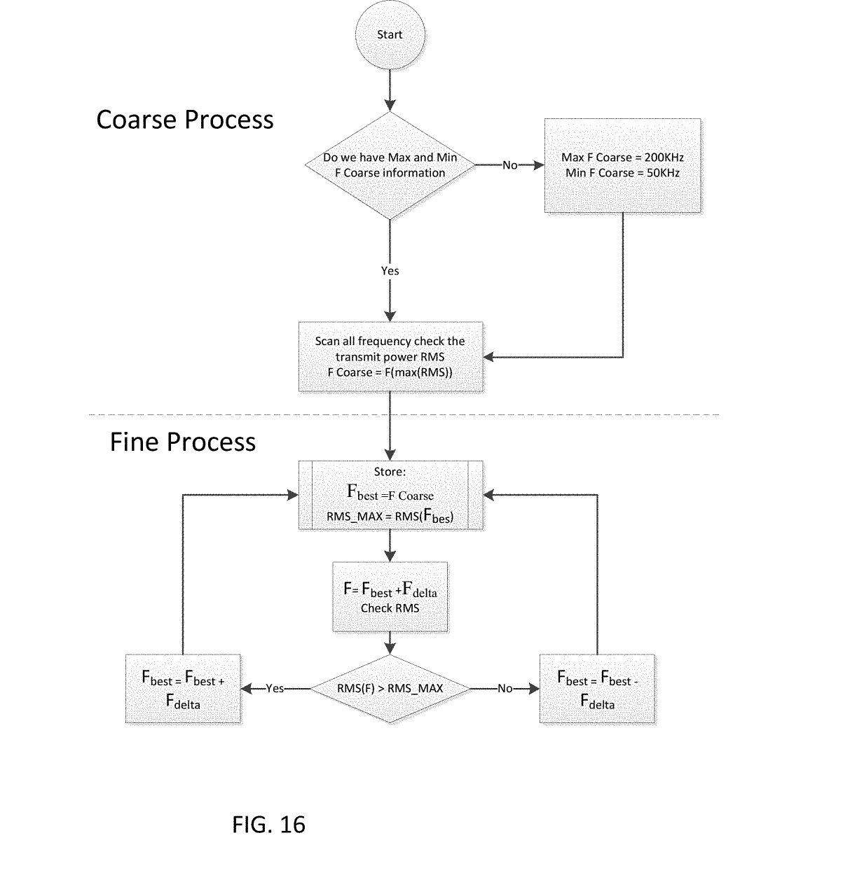

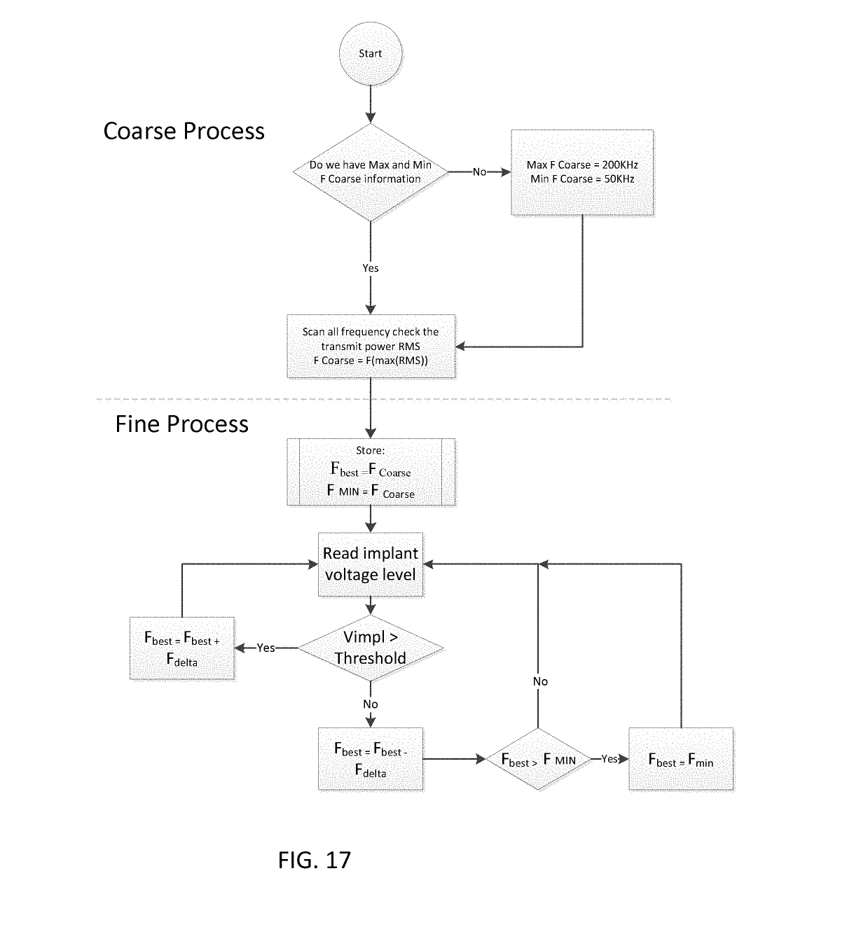

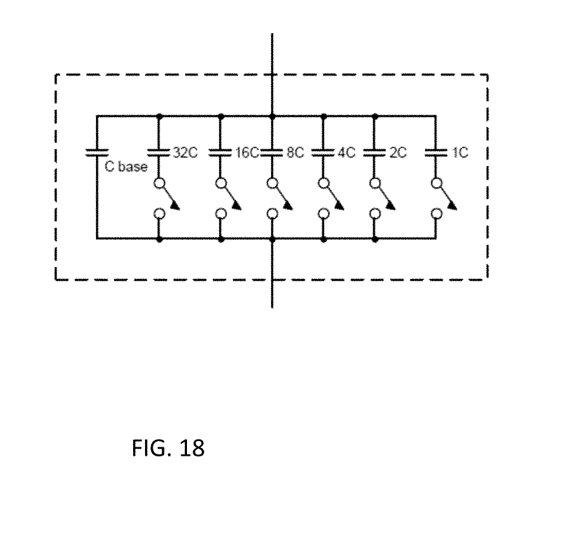

As described in FIG. 16 and in FIG. 17 the transmitter can detect the receiver's resonance frequency in two phases. First, in a coarse phase, when no pre-detected frequency is available, the transmitter uses a fast frequency detection process to roughly detect the receiver resonance frequency or else just start at some predetermined frequency. Second, in a fine phase that occurs after the coarse phase, the transmitter uses an ongoing process of fine tuning to detect the receiver resonance frequency.

The main difference between the two procedures is the simplicity of the solution. FIG. 17 describes a very simple system where the coarse phase detects roughly the resonance, which then becomes the minimum frequency limit. (The system doesn't use the resonance frequency exactly, it uses a frequency above (or below) the resonance and then controls the transfer power by tuning the frequency). This is a simple system and it can work in strong coupling environment like the CET system. In other instances, when the coupling is lower due to distance or receiver/transmitter size/quality it is necessary to use the exact resonance frequency to be able to transfer the needed power.

FIG. 16 describes the fine process that occurs after the first coarse adjust approximately determines the transmitter resonance. In the coarse phase, a microcontroller (MCU) associated with the transmitter resonance structure can have preliminary information about the receiver resonance frequency. The MCU will change the transmitter's driver frequency one after the other and detect the root mean square (RMS) current in the one or more coils of the transmitter. At the end of this phase, the MCU has the result of the entire frequency spectrum, and it can automatically select (as a result of its software programming) the best first coarse frequency, F_{coarse}.

After the coarse phase, the fine phase begins, in which the MCU's software programming dictates the selected frequency from the coarse phase as the best known resonance, F_{best}. Once in the fine phase, the MCU stores the RMS current, adds single F_{delta} to the previous frequency and stores that RMS current. By comparing these two RMS currents, the transmitter's MCU determines whether to add F_{delta} or to reduce F_{delta} from the previous F_{best}. The equation used is as follows: F_{best}=F_{best}+/−F_{delta}. Then, the transmitters' resonance frequency is locked to the receiver's resonance frequency until the next fine phase process occurs. The fine phase process can occur periodically every T_{fine}.

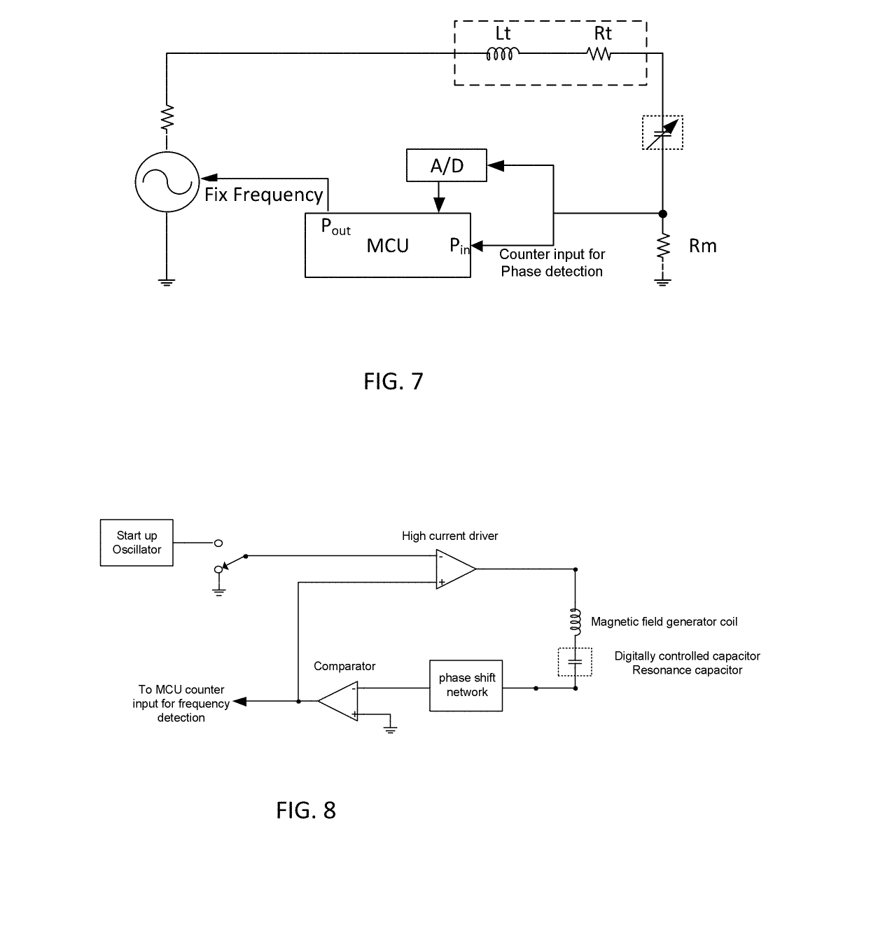

Locking the transmitter resonance to the detected receiver resonance involves the transmitter coil automatically adjusting its capacitors, which can be accomplished using either the circuit shown in FIG. 7, or the circuit shown in FIG. 8, each of which is a resonance LC (inductance and capacitance) structure.

In the circuit of FIG. 7, the MCU forces a fix frequency by generating Pₒᵤₜ pulse in the requested frequency and push the driver circuit. In so doing, the MCU thereby calibrates the configurable capacitor to get resonance. The MCU receives the feedback phase, and adjusts it to the resonance. In resonance, the feedback phase Pᵢₙ should be exact as the generated one Pₒᵤₜ. The MCU then compares the output Pₒᵤₜ to the input Pᵢₙ to validate the resonance. The MCU should adjust the capacitors according to the phase until Pᵢₙ=Pₒᵤₜ

In the circuit of FIG. 8, the circuit is a self-oscillating circuit, and thus is always in resonance; however the MCU can adjust the frequency by changing the capacitors. The MCU can add capacitors to the capacitors array or remove capacitors as described in FIG. 16.

Although FIGS. 7 and 8 show two particular circuits that can be used, it is noted that a variety of variants of phase locked loop (PLL) algorithms and implementing circuits can be used to compensate for impedance changes of the coils by adjusting capacitor value.

### Optimizing Frequency of the Power Transfer:

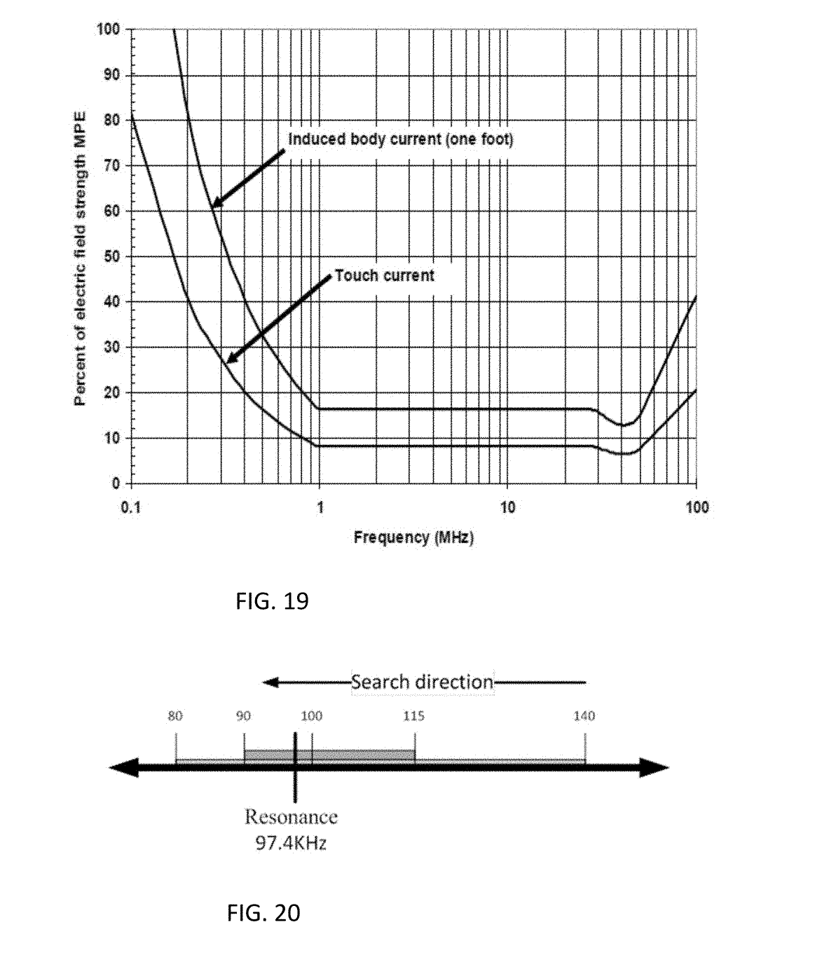

Wireless energy transfer to an implanted medical device, e.g. CET, requires consideration of two parameters, namely (1) the effect of wireless energy transmission on the living tissue through which it is transmitted and (2) the loss in efficiency of the wireless energy transfer due absorption by the living tissue. Other limitations of energy transfer are energy ranges deemed injurious to tissue, as in the ranges set forth in the IEEE Standard C95.1. For example, ranges 3 kHz to 5 MHz can cause painful electro stimulation; 100 kHz to 300 GHz can cause tissue heating (as shown in FIG. 19); and 100 kHz to 5 MHz can cause both electro stimulation and tissue heating. With those parameters and limitations taken into consideration, the optimal frequency of power transmission between a transmitter coil and a receiver coil of the invention provide for high power efficiency and high power transmission without any associated undesirable tissue heating. FIG. 19 shows a percent of electric field strength MPE in the induced body current (of one foot) and the touch current at certain frequencies.

CET systems of the invention achieve optimal energy transfer (such as electromagnetic power) by employing frequencies ranging from 60 KHz to 1 MHz. In that range, the preferred frequency may range from 80 KHz to 300 KHz. In other embodiments, the preferred frequency may range from 90 KHz to 115 KHz. Using frequencies within that range, systems of the invention provide for high power efficiency and high power transmission without any associated undesirable tissue heating.

CET systems of the invention may be programmed to search for a target frequency in order to optimize the functional conditions. This search may be automatic. For the search, the system may utilize circuitry—e.g. a microcontroller (e.g., as in FIGS. 7 and 8), a phase-locked loop circuit (as with analog circuits), or other processor—associated with the transmitter to search and detect the certain frequency of the receiver coil and lock the frequency of the transmitter coil to the receiver coil. The search circuitry controls the transmitter frequency according to input. Depending on the application, the certain frequency may be the resonance frequency or a non-resonance frequency.

The search circuitry may utilize several loop input measurements in order to detect the certain frequency. In addition, the search circuitry may rely on the same principles discussed above to detect the frequency of the receiver and lock the frequency of the transmitter to the receiver. The loop input measurements include, for example, the transmitter power, the receiver power, or other parameters such as an implant current parameter, an implant voltage parameter, an implant charging parameter, a hit parameter, etc. When utilizing transmitter power as an input measurement, the system detects the mutual resonance frequency of the transmitter and receiver at the point where the current in the transmitter inductive coil is maximized. In embodiments that utilize the transmitter power, the circuitry required for the search can be self-contained on the transmitter, and no communication is required from the receiver. When utilizing receiver power as an input measurement, the system detects the mutual resonance frequency of transmitter and receiver at the point where the power within the receiver is maximized, which can be sensed from the current with or without voltage in the receiver circuit. In embodiments that utilize the receiver power or receiver parameters for the search, a microcontroller or other circuitry should also be associated with the receiver to obtain measurements and allow for communication from the receiver to the transmitter.

For resonance frequency searching, the search is initiated by the transmitter circuit. The search utilizes a loop to locate the best frequency with one of the input measurements based on the transmitted efficiency, which is indicated by the current in the transmitter. The search goes down (or up) a range of frequencies (such as the dynamic band), and the resonance frequency is detected because it provides peak efficiency. That is frequencies before and after the resonance frequency are not as efficient, thereby allowing the system to detect the resonance frequency.

For non-resonance frequency searching, the search is initiated by the transmitter circuit. The search utilizes a loop to locate a target frequency with one of the input measurements based on the transmitted efficiency, which is indicated by the current in the transmitter. The search goes down (or up) a range of frequencies, and will stop when it reaches the desired efficiency. By not operating on the resonance frequency, the system is able to control power by manipulating the frequency. For instance, in some CET systems, the desired frequency is 98 KHz just above ideal the resonance frequency of 97.4 KHz. This allows power transfer control simply by changing the frequency. In those instances, the system transmits high power when using a frequency near the resonance and low power when using frequencies farther from resonance.

The resonance and non-resonance frequency searches may be conducted across a dynamic band of frequencies. The dynamic band is the general search range of frequencies. The search typically ends at lowest frequency in the dynamic band. The dynamic band may range from 80 KHz to 140 KHz. The lowest frequency in the dynamic range may be a frequency where no resonance is found. If a system reaches the lowest frequency within the band without finding a resonance frequency of the receiver, the system may terminate the search and may trigger an alarm sound. For example, if 80 KHz is the lowest frequency in the dynamic band, the system will search within the frequency range from the highest frequency to the lowest frequency. In certain embodiments, the search will terminate and an error alarm will sound if the system search reaches the lowest frequency of 80 KHz without finding the resonance frequency of the receiver.

Typically, resonance and non-resonance frequency searches will start at a higher frequency of a dynamic band (e.g., about 140 KHz), and then adjust the search downward towards the resonance or target frequency until the resonance or target frequency is detected. For non-resonance searching, the target frequency may be within range of frequencies called the target main frequencies, which are frequencies desired and targeted by the CET system for optimal efficiency. In certain embodiments, the target main frequency range is from 90 KHz to 115 KHz. It is understood that other frequencies can be used as for the dynamic band and target main frequencies depending on the application (e.g. depending on the selected frequency range).

The following describes using the dynamic band to search for a resonance or target frequency in accordance with FIG. 20. Circuitry associated with the transmitter conducts a search of a dynamic band of frequencies ranging from 140 KHz to 80 KHz. The dynamic band of frequency overlaps with the range of target main frequencies (ranging from 90 KHz to 115 KHz), with the resonance frequency being 97.4 KHz. The transmitter will start search at the top of dynamic band at 140 KHz and then adjust to lower frequencies until the resonance frequency is found or a target frequency within the range of target main frequencies is reached. When conducting a resonance search, the system will initiate an alarm if the search does not find the resonance frequency when it reaches the lower limit of about 80 KHz.

### Placement in a Patient's Body of the Receiver Resonance Structure:

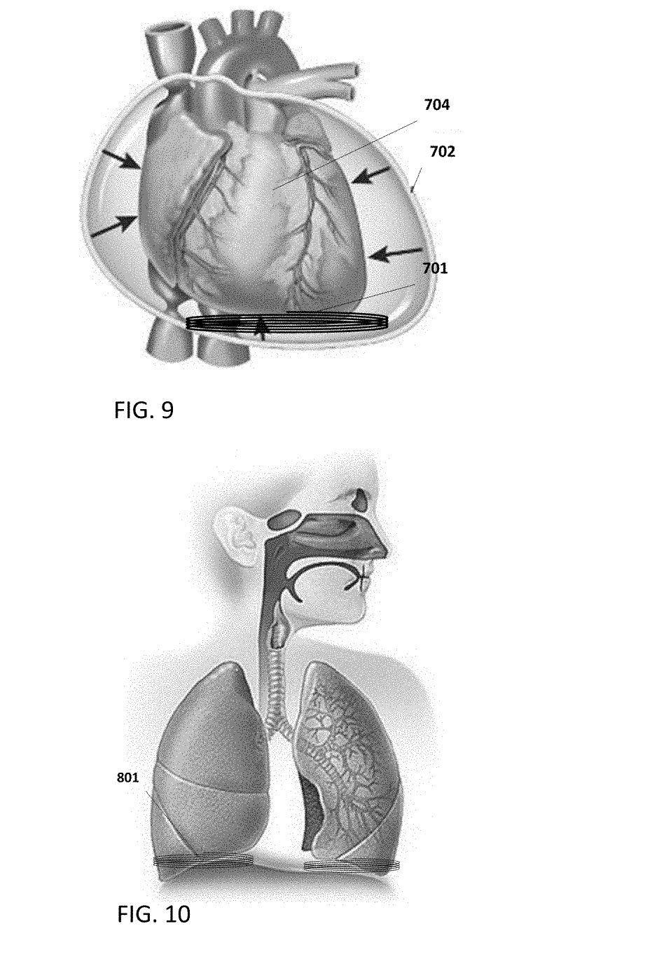

The receiver inductive coil can be placed within the body of a patient at a variety of internal locations. FIGS. 9, 10, and 11 illustrate three particular examples of a placement location inside the body of a patient.

As shown in FIG. 9, the receiver coil 701 may be placed in the base of the flat part of the pericardia 702, which surrounds the heart 704. The main added value in placing the receiver coil 701 in the pericardia 702 with a VAD is that the pericardia 702 is relatively flat and open in typical VAD surgery. The receiver coil 701 can be glued to the pericardia 702 boundaries, e.g., with surgical glue.

In FIG. 10, it is shown that the receiver coil 801 of a VAD can be placed in the pulmonary cage. One advantage of placing the coil 801 in the pulmonary cage is that the VAD will not disturb the magnetic power harvesting, and that pulmonary cage is relatively easy to access during the VAD surgery.

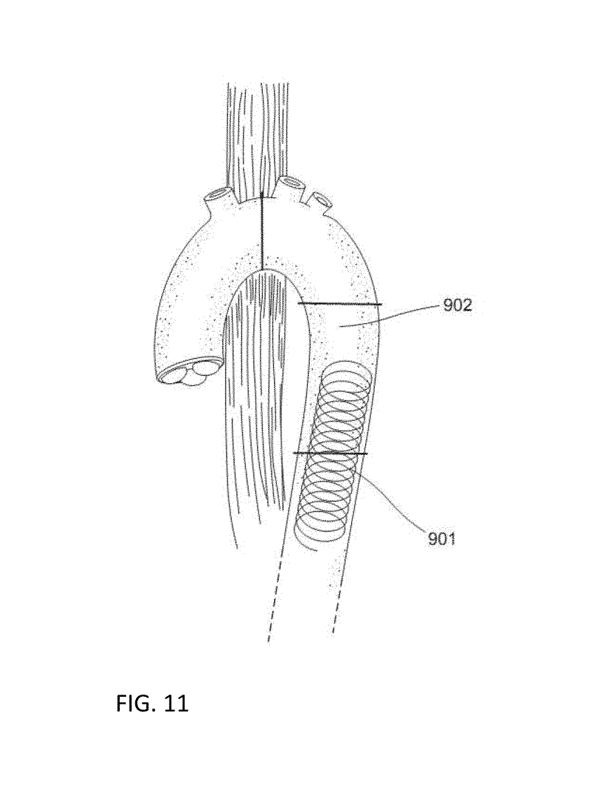

As shown in FIG. 11, the receiver coil 901 may also be placed in an artery 902. The Aorta or the Vena Cava are particularly well-suited for placement of the receiver coil 901 because each is oriented vertically with respect to a plane that cuts in a cross section through the torso of the patient. Placement of the receiver coil 901 in the Aorta or the Vena Cava also allows the receiver coil 901 to be associated with an implantable stent.

### Further Disclosure Related to Providing an Optimum Load to the Receiver Resonance Structure:

Having presented various details of various embodiments according to the invention, some theory, equations, and calculations relevant to providing an optimum load to the receiver resonance structure will now be presented.

The ratio between the distance D from the transmitting coil to receiving coil and the wavelength λ is as follows:

$$\frac{D}{λ} = \frac{Df}{c} , \qquad ( 1 )$$

where f is the transmitting frequency and c=3·10⁸ m/s is the speed of light.

Given that the maximum distance Dₘₐₓ does not exceed 0.4 m and the working frequency is f=100 kHz, the ratio Dₘₐₓ/λ=0.00013<<1. Thus, we can conclude that the receiving coil is in the quasi-static area, and we can neglect the effects of the phase difference due to the wave propagation.

The amplitude of the voltage induced in the receiving coil according to the Faraday's law [1] is as follows:

$${v}_{r} ( t ) = - \frac{d Φ}{dt} = - \frac{d}{dt} ( B \cdot a ) , \qquad ( 2 )$$

where Φ is the magnetic flux through the receiving coil, B is the magnetic flux density, and a is the effective area of the receiving coil.

To estimate the maximum induced voltage (2), assume that the receiving coil is located coaxially with the transmitting coil at its center, where the magnetic flux density B can be calculated as follows [1]:

$$B = \frac{{μ}_{r} {μ}_{0} {I}_{t} {N}_{t}}{2 {R}_{t}} sin ( 2 π ft ) , \qquad ( 3 )$$

where μᵣ is the relative permeability of media, μ₀=4π10⁷ V·s/(A·m) is the permeability of vacuum, Iₜ is the amplitude of the current in the transmitting coil, and Rₜ and Nₜ are the radius and number of turns of the transmitting coil correspondingly.

The effective area of the receiving coil can be calculated as follows:

a=πRᵣ²Nᵣ, (4)

where Rᵣ and Nᵣ are the radius and the number of turns of the receiving coil correspondingly.

Substituting (3) and (4) into (2) and differentiating with respect to the time, gives the following expression for the amplitude of the voltage induced in the receiving coil:

$${V}_{r} = 2 π f \frac{{μ}_{r} {μ}_{0} {I}_{t} {N}_{t}}{2 {R}_{t}} π {R}_{r}^{2} {N}_{r} . \qquad ( 5 )$$

The transmitting and the receiving coils can be seen as two coupled inductors, as follows:

$$\{ \begin{matrix}{v}_{t} = {L}_{t} \frac{{di}_{t}}{dt} - M \frac{{di}_{r}}{dt} \\ {v}_{r} = - M \frac{{di}_{t}}{dt} + {L}_{r} \frac{{di}_{r}}{dt}\end{matrix} , \qquad ( 6 )$$

where vₜ and vᵣ are the transmitter and receiver coils voltages, iₜ and iᵣ their currents, and M is the mutual inductance.

Assuming that the current in both coils is a sine-wave of frequency ω=2πf, (6) can be written as follows:

$$\{ \begin{matrix}{v}_{t} = j ω {L}_{t} {i}_{t} - j ω {Mi}_{r} \\ {v}_{r} = - j ω {Mi}_{t} + j ω {L}_{r} {i}_{r}\end{matrix} . \qquad ( 7 )$$

The mutual inductance M can be found from the open circuit experiment, where iᵣ=0:

$$\{ \begin{matrix}{v}_{t} {\vert}_{{i}_{r} = 0} = j ω {L}_{t} {i}_{t} \\ {v}_{r} {\vert}_{{i}_{r} = 0} = - j ω {Mi}_{t}\end{matrix} . \qquad ( 8 )$$

Rearranging the second equation of (8) with respect to M and substituting (2)-(5) gives us:

$$M {\vert}_{{i}_{r} = 0} = - \frac{{v}_{r}}{j ω {i}_{t}} = - \frac{- \frac{d}{dt} ( B \cdot a )}{j ω {i}_{t}} = \frac{{μ}_{r} {μ}_{0} {N}_{t}}{2 {R}_{t}} π {R}_{r}^{2} {N}_{r} = 3.9 µH . \qquad ( 9 )$$

The value of M obtained in (9) increases as a function of the relative permeability μᵣ of the receiver core.

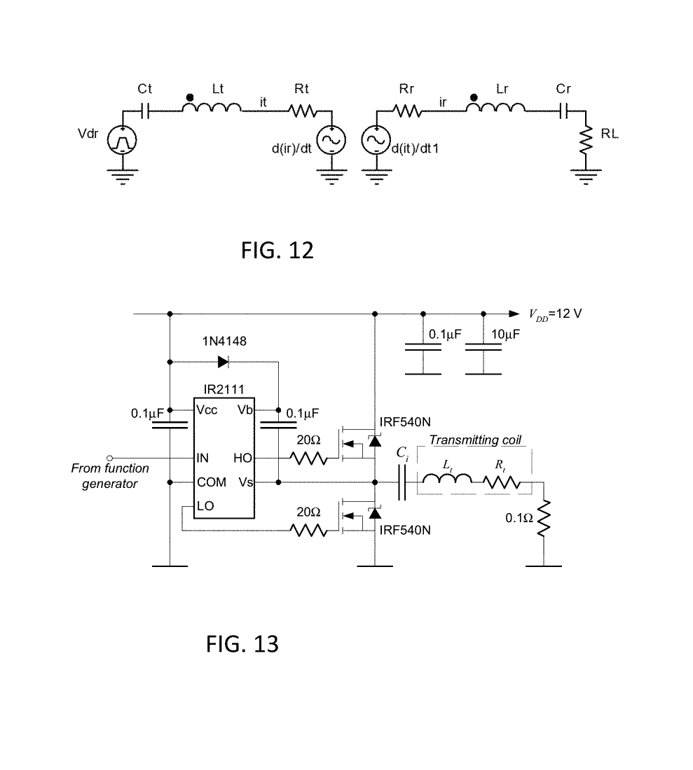

For the purpose of efficiency calculation, assume that the transmitter coil is loaded with a series resonant capacitor and the receiver coil is loaded to form a series resonant circuit as describe in FIG. 12.

The transmitter current is calculated using the coupled-inductor model (7), as follows:

$${i}_{t} = \frac{{v}_{s}}{{R}_{t} + \frac{{( ω M )}^{2}}{{R}_{r} + {R}_{L}}} , \qquad ( 24 )$$

where vₛ=2V_{DD}/π is the effective voltage of the source V_{dr} at the first harmonic of the excitation frequency, Rₜ is the active resistance of the transmitter coil, Rᵣ the active resistance of the receiver coil, and R_{L} is the load resistance.

The amplitude of the load voltage is given by:

$${V}_{L} = \frac{2 ω {MV}_{DD} / π}{{R}_{t} + \frac{{( ω M )}^{2}}{{R}_{r} + {R}_{L}}} \cdot \frac{{R}_{L}}{{R}_{r} + {R}_{L}} = \frac{2 ω {MV}_{DD} / π}{{R}_{t} ( {R}_{r} + {R}_{L} ) + {( ω M )}^{2}} \cdot {R}_{L} , \qquad ( 25 )$$

where V_{DD} is the supply voltage of the half-bridge driver of the transmitter.

From here, the load power is given by:

$${P}_{L} = \frac{{V}_{L}^{2}}{2 {R}_{L}} = \frac{2 {( ω {MV}_{DD} / π )}^{2} {R}_{L}}{{( {R}_{t} + ( {R}_{r} + {R}_{L} ) + {( ω M )}^{2} )}^{2}} \cdot \qquad ( 26 )$$

Differentiating (26) with respect to R_{L} gives the load resistance that maximizes the load power, as follows:

$${R}_{Lopt} = {R}_{r} + \frac{{( ω M )}^{2}}{{R}_{t}} . \qquad ( 27 )$$

Substituting (27) into (26) yields:

$${P}_{Lopt} = 0.5 \frac{{( {V}_{DD} / π )}^{2} {( ω M )}^{2} / {R}_{t}}{{R}_{t} {R}_{r} + {( ω M )}^{2}} . \qquad ( 28 )$$

Rearranging (28) with respect to the driver voltage gives:

$${V}_{DD} = \frac{π}{ω M} \sqrt{2 {P}_{Lopt} {R}_{t} ( {R}_{t} {R}_{r} + {( ω M )}^{2} ) .} \qquad ( 29 )$$

The input power is:

$${P}_{t} = \frac{{V}_{DD}}{2 π} {\int }_{0}^{π / ω} {i}_{t} ( t ) dt = 2 {( \frac{{V}_{DD}}{ π} )}^{2} \frac{{R}_{r} + {R}_{L}}{{R}_{t} ( {R}_{r} + {R}_{L} ) + {( ω M )}^{2}} , \qquad ( 30 )$$

while its optimal value considering (27) is:

$${P}_{topt} = {( \frac{{V}_{DD}}{ π} )}^{2} \frac{2 {R}_{r} + {( ω M )}^{2} / {R}_{t}}{{R}_{t} {R}_{r} + {( ω M )}^{2}} . \qquad ( 31 )$$

Dividing (29) by (31) gives the efficiency of the wireless power transmission corresponding to the optimum load resistance:

$${η}_{opt} = \frac{{P}_{Lopt}}{{P}_{topt}} = 0.5 \frac{1}{1 + \frac{2 {R}_{r} {R}_{t}}{{( ω M )}^{2}}} . \qquad ( 32 )$$

The general expression for the efficiency is:

$$η = \frac{{P}_{L}}{{P}_{t}} = \frac{{( ω M )}^{2}}{{R}_{t} ( {R}_{r} + {R}_{L} ) + {( ω M )}^{2}} \cdot \frac{{R}_{L}}{{R}_{r} + {R}_{L}} . \qquad ( 33 )$$

Differentiating (33) with respect to R_{L} gives the load resistance that maximizes the efficiency:

$${R}_{L η max} = \sqrt{{R}_{r}^{2} + \frac{{( ω M )}^{2} {R}_{r}}{{R}_{t}}} . \qquad ( 34 )$$

The maximum efficiency can be calculated by substituting (34) into (33).

$${η}_{max} = 0.5 \frac{1}{1 + \frac{2 {R}_{r} {R}_{t}}{{( ω M )}^{2}}} . \qquad ( 32 )$$

The maximum efficiency and maximum load power for the parallel-loaded receiver is identical to that of the series one. The optimal load resistance and maximizing the efficiency for the parallel-loaded receiver differ from (27) and (34). However, the derivation is similar. The specific formulae for the load resistance is not developed here and instead we find the optimal resistance using computer simulations tool like PSPICE® (Cadence Design Systems, San Jose, Calif.), a full-featured, native analog and mixed-signal circuit simulation tool.

The circuit shown in FIG. 13 is a transmitter. The source Vdr is built from two BUZ11 N-MOSFETs driven by the IR2111 gate driver. The 0.1 Ohm resistor is used for the transmitter current monitoring. Both the transmitter and receiver capacitors are chosen with low ESR. The load resistance is chosen as R_{L}=0.5 Ohm, and the driver voltage V_{DD}=12 V. Substituting these values and the other setup parameters (Rₜ=1 Ohm, Rᵣ=0.65 Ohm, M=2.056 μH) into (26) gives for P_{L}=3.16 W. The measured voltage amplitude on the load resistance is 1.75 V, which corresponds to P_{L}=3.1 W. The input power drawn from the power supply is Pᵢₙ=V_{DD}/π·I=12/3.14·2.8=10.7 W. The efficiency is η=P_{I}/Pᵢₙ=28%. It is noted that the load resistance is not optimized for the maximum output power.

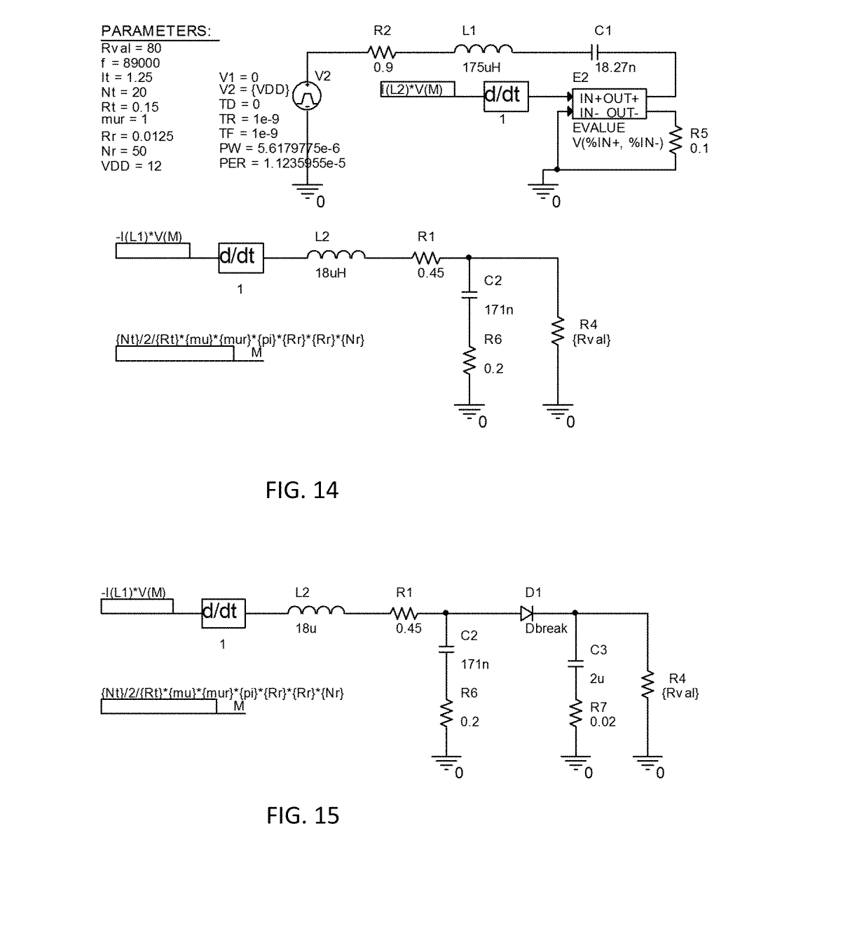

The circuit shown in FIG. 14 is a parallel-loaded receiver. The source V2 is built from two BUZ11 N-MOSFETs driven by the IR2111 gate driver. The 0.1 Ohm resistor is used for the current monitoring. Both the transmitter and receiver capacitors are chosen with low ESR. Substituting the model parameters into (29) gives V_{DD}=11.5 V for P_{L}=5 W. Computer simulations have shown that the maximum load power of 4.85 W is obtained for R_{L}=80 Ohm. This result closely correlates with laboratory measurements, where an output power of 4.5 W was measured for V_{DD}=12 V. The input power drawn from the power supply is Pᵢₙ=V_{DD}/π·I=12/3.14·4.2=16.05 W. The efficiency is η=P_{I}/Pᵢₙ=28%.

Inserting a simple single-phase rectifying circuit before R4, as shown in the circuit in FIG. 15, takes about 0.2 W dissipated on the diode with 2 A peak diode current and 44 V peak diode reverse voltage. The peak voltage on the receiver capacitor is 25 V, and the peak voltage on the transmitter capacitor is 500 V.

### Optimizing the Design of the Resonance Structure of the Transmitter and Receiver

Several design factors contribute to the quality of the transmitter and receiver resonance structures of CET systems of the invention. Particularly, the resonance structure (e.g. resonance structure 202 as shown for the Receiver in FIG. 4) can be designed to minimize loss during the energy transfer. The quality of the resonance structure is dependent on the ratio between the inductance (L) to the resistance (R) of the coil. In radio frequency (RF) couplings, the quality of the resonance LC structure is dependent on the ratio of the inductance and capacitance (LC) to the resistance (R) of the coil. Those ratios are referred to herein as the quality factor (Q). Resonance LC structures are depicted in FIGS. 7 and 8. A higher Q indicates a lower rate of energy loss relative to the stored energy of the resonance structure. The quality factor (Q) of the coil originates in the coil's ohmic resistance, which can be calculated knowing the material and thickness of the coil, the diameter of the coil, D, the number of turns of coil, N, and the magnetic effects—the skin effect and the proximity effect (described below). In addition to the regular resistance and loss factors, a design may also take into consideration the skin effect and the proximity effect and the capacitor internal resistance.

The quality factor Q of the receiver and/or transmitter resonance structures can vary to provide optimum energy transfer. In certain embodiments, the quality factor of the transmitter is within the range of about 100 to about 500, and the quality factor of the receiver is within the range of about 50 to about 200.

In certain embodiments, the quality factor of the coils is improved with nongalvanic connected coils.

The following describes the various parameters that influence the Q of RF couplings.

The first parameter is a capacitor's or capacitors' equivalence series resistance (ESR) of the resonance structure of either the transmitter or receiver coils. For optimal Q, a capacitor's or capacitors' ESR in a resonance structure is less than the coil's active resistance. In certain embodiments, the capacitor's or capacitors' ESR is less than 5 times the coil's active resistance.

Another parameter that influences Q is the skin effect. The skin effect is the tendency of an alternating electric current (AC) to become distributed within a conductor such that the current density is largest near the surface of the conductor, and decreases with greater depths in the conductor. That is, the electric current flows mainly at the “skin” of the conductor, between the outer surface and a level called the skin depth. The skin effect causes the effective resistance of the conductor to increase at higher frequencies where the skin depth is smaller, thus reducing the effective cross-section of the conductor.

In order to reduce the skin effect in transmitter and receiver coils, the coils can be constructed using wires configured to transmit alternating currents, such as litz wire. A litz wire is a type of cable used in electronics to carry alternating current, and are made according to the “litz wire standards.” Litz wires consist of many thin wire strands, individually insulated and twisted or woven together, following one of several known patterns often involving several levels (groups of twisted wires are twisted together, etc.). Litz wires suitable for use in systems of the invention include those manufactured by New England Wire Technologies (Libson, NH). Litz wire standards relate the number of internal strands to the wire structure. Litz wire sizes are often expressed in abbreviated format: N/XX, where N equals the number of strands and XX is the gauge of each strand in AWG (American Wire Gauge). Wires suitable for use in the invention have a gauge of 36-48 AWG (with the preferred gauge being 38-40 AWG). In certain embodiments, the external coil is a wire with 100-600 strands with a gauge of 36-48 AWG; and the internal coil is a wire with 100-400 strands with a gauge of 36-48 AWG. In preferred embodiments, the internal coil and the external coil are formed from a wire with 175 strands/40 AWG.

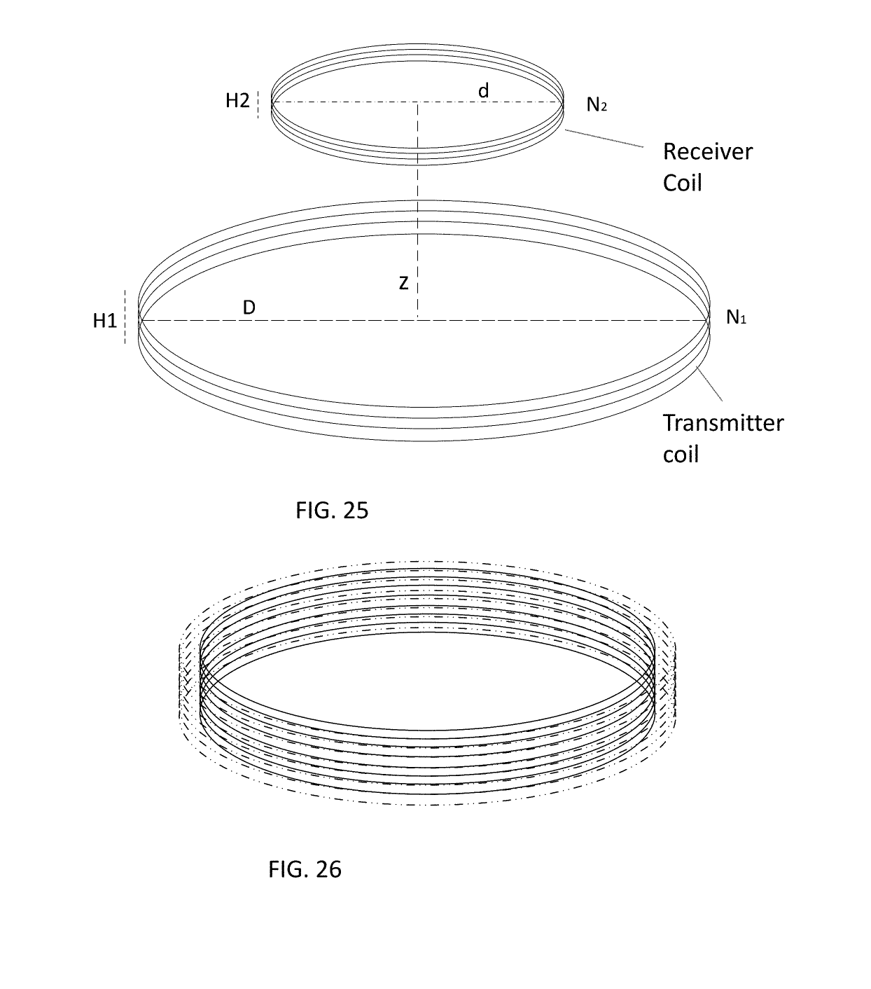

Another parameter that influences Q is the proximity effect. Proximity effect is the tendency for current to flow in loops or concentrated distributions due to the presence of magnetic fields generated by nearby conductors. The proximity effect is most evident in a conductor carrying alternating current, if currents are flowing through one or more other nearby conductors, such as within a closely wound coil of wire, the distribution of current within the first conductor will be constrained (or crowded into) to smaller regions. This current crowding is known as the proximity effect. This crowding gives an increase in the effective resistance of the circuit. The resistance due to the proximity effect increases with frequency. The proximity effect, in transmission and receiver coils relates to H1, H2 and coil center to center distance z (see FIG. 25). In the end, the proximity effect cannot be uncoupled from geometry, and must be calculated for a given design.

The following are additional optimization features that can also be incorporated into the transmitter and receiver of the CET systems of the invention.

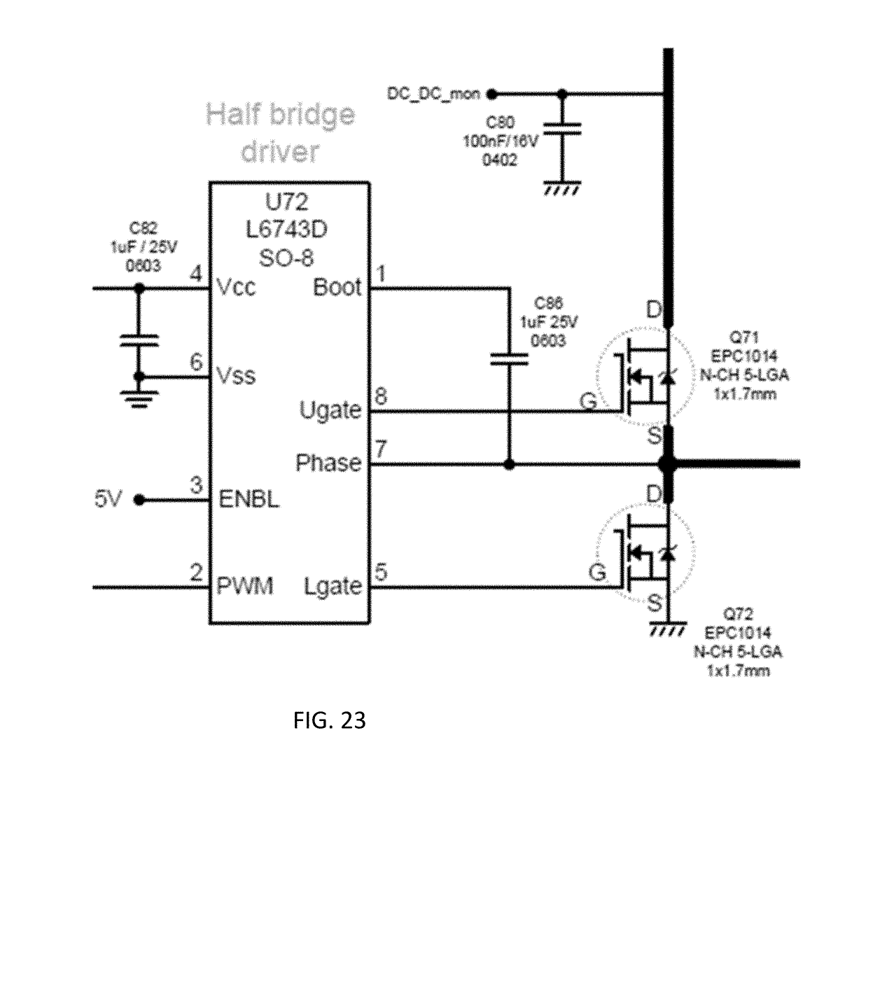

In certain embodiments, the transmitter is optimized by incorporating a square generator, such as a half bridge pulse generator. The half-bridge pulse generator assists with pushing power from the transmitter to the receiver. FIG. 23 depicts a preferred circuit for to half bridge pulse generator. While the circuit depicted in FIG. 23 is a square wave generator, the circuit can generate regular sinusoidal waves in the transmitter coils.

Another optimization feature includes the incorporation of a field-effect transistor in the receiver and/or transmitter. A field-effect transistor (FET) is a transistor that uses an electric field to control the shape and hence the conductivity of a channel of one type of charge carrier in a semiconductor material. Particularly, FETs with low resistance when in saturation can greatly reduce losses due to heating.

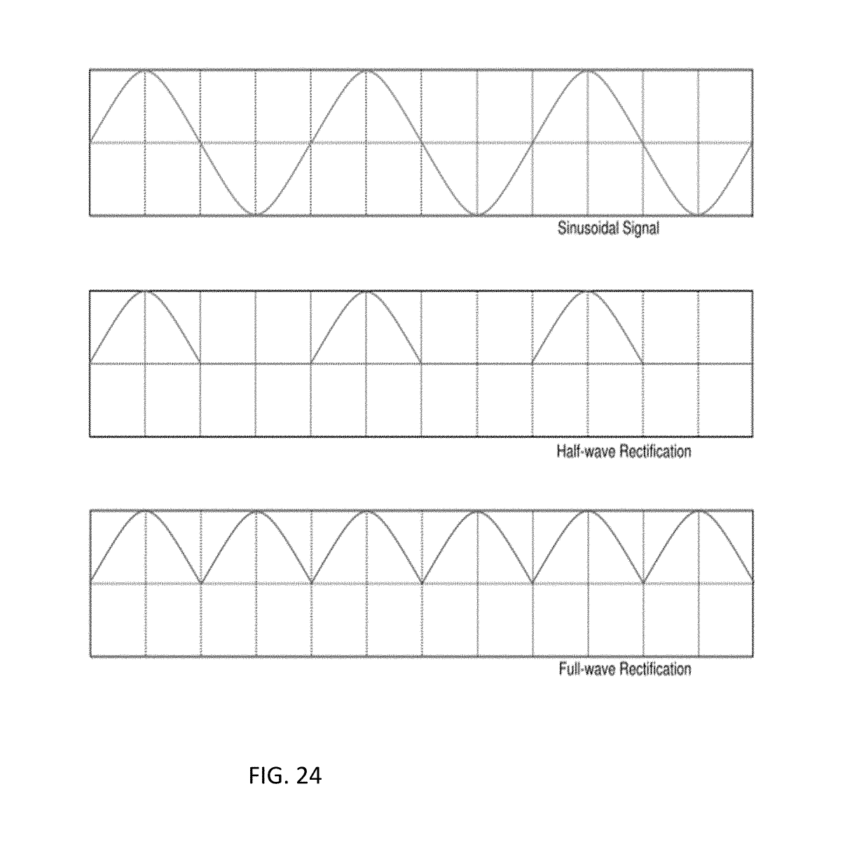

In addition, an AC-to-DC conversion circuit may be included in the receiver in order to convert the AC voltage from an AC power source to DC voltage. The AC-to-DC conversion is a process known as rectification. In some embodiments, diodes can be utilized in systems of the invention to reduce loses in the AC to DC conversion circuit. Regular AC to DC conversions involve diode-based rectification circuits. Any rectifier may be used to convert AC voltage to DC current. In certain embodiments, CET systems may utilize a single diode rectification circuit or a diode bridge rectification circuit with the receiver. FIG. 24 illustrates rectification with one diode (half wave) and with a diode bridge (full wave). A diode bridge is an arrangement of four (or more) diodes in a bridge circuit configuration that provides the same polarity of output for either polarity of input. The advantage of the diode bridge is that it uses the entire input wave rather than only half of it.

According to some embodiments, the receiver includes a single diode rectifier. The effect of the single diode will be the same as using a diode bridge because the energy of the closed cycle of the single diode is not lost. Instead, the energy is kept the resonance structure for the active cycle. By using a single diode, only part of the duty cycle is used, and the potential difference in the resonance circuit will be significantly higher than built-in voltage drop across the diodes (around 0.7 V for ordinary silicon p-n junction diodes and 0.3 V for Schottky diodes). Thus, a single diode is able to reduce the transaction. In addition, the ratio of the diode's built-in voltage drop to the total voltage will be better. Also, the efficiency of the diode is related to the diode's size. Therefore, a system utilizing one diode in a rectifier of a certain size is more efficient than four diodes of the same size. In certain embodiments, the diode is a Schottky diode. Schottky diodes minimize the transition cycle loss.

### Effect of a Coplanar Wireless Energy Transfer System's Design and Geometry on Power Transmission

The geometry of the CET systems can affect the efficiency and robustness of the system. Accordingly, the geometry of certain systems can be optimized in accordance with the medical device being powered by a CET system. For example, the type of medical device, its power needs, and the implantation site are inputs that can be used to shape the geometry of a system for optimal energy transfer.

The following are key parameters that effect geometry and design of a CET system of the invention for use with an implant, such as a ventricular assist device (VAD). These systems include generally transmission of power from a belt or a vest transmitter that circumscribes the body in the same plane of the receiver. Optimal key parameters are suggested for and based on a typical VAD having a power requirement of 5 W-20 W and a peak of 30 W. In addition, the optimal key parameters for transmitters and receivers for use with a typical VAD are described in further detail separately below.

FIG. 25 depicts a layout of a CET system for use with a VAD. This layout is helpful for understanding geometry dependent factors. The distance Z, as shown in FIG. 25, is the distance between the centers of the transmitter and receiver coils. In general, the smaller the distance Z is, the better the coupling between the transmitter and the receiver. Preferably, the distance Z is 7 cm or less. Ideally, the receiver coils are concentric with the transmitter coils such that the distance Z is minimized. However, systems described herein maintain power transfer efficiency while allowing distance Z of about 7 cm between the centers of the transmitter and the receiver.

In addition, the diameters of transmitter and receiver coils (see FIG. 25) effect power transmission in a system. Diameter D is the diameter of the external transmitter, which depends and can be adjusted based on the body size of a patient. Diameter d is diameter of the internal receiver. The diameter d of the internal transmitter can be varied depending on the type and placement of the device. As diameter d increases, so does the quality of the coupling between the transmitter and receiver. The ratio of the receiver diameter d to the transmitter diameter D also affects wireless power transmission. In general, a higher diameter ratio increases the quality of the coupling for energy transfer.

Further, the number of wire turns of the transmitter coil N1 and receiver coil N2 also influences power transmission. The greater number of turns of wire in either coil improves magnetic/electronic conversion. However, the resistance caused by the number of wire turns should also be taken into consideration.

Other design considerations that influence power transmission are the capacitor's ESR and the type of wire used (both of which were discussed in further detail above). For optimization, the capacitor's ESR should be as low as practical, and a litz wire should be used to minimize skin effect.

A. Transmitter Geometry and Design

The following are preferable design and geometry details of the transmitter.

According to certain embodiments, the diameter of the transmitter coil D, as shown in FIG. 25, can be sized to fit around an individual's body. In certain embodiments, the diameter D ranges from, for example, about 20 cm (for children) to about 60 cm (overweight adult).

In certain embodiments, the transmit frequency may be in the range of about 60 KHz to about 1 MHz. As discussed in more detail above, the CET system can be designed to search across a range of frequencies such that the transmitter couples to the resonance frequency of the receiver. This search may be automatic. For automatic frequency searches, the range of frequencies searched (e.g. a dynamic band of frequencies) are narrower than the range of the transmit frequency. In addition, a transmitter may be set at a target frequency or a resonance frequency. The dynamic band may be between 80 KHz and 300 KHz. In other embodiments, the dynamic band may be 80 KHz to 140 KHz. The target frequency may be a frequency within the range of 90 KHz to 115 KHz.

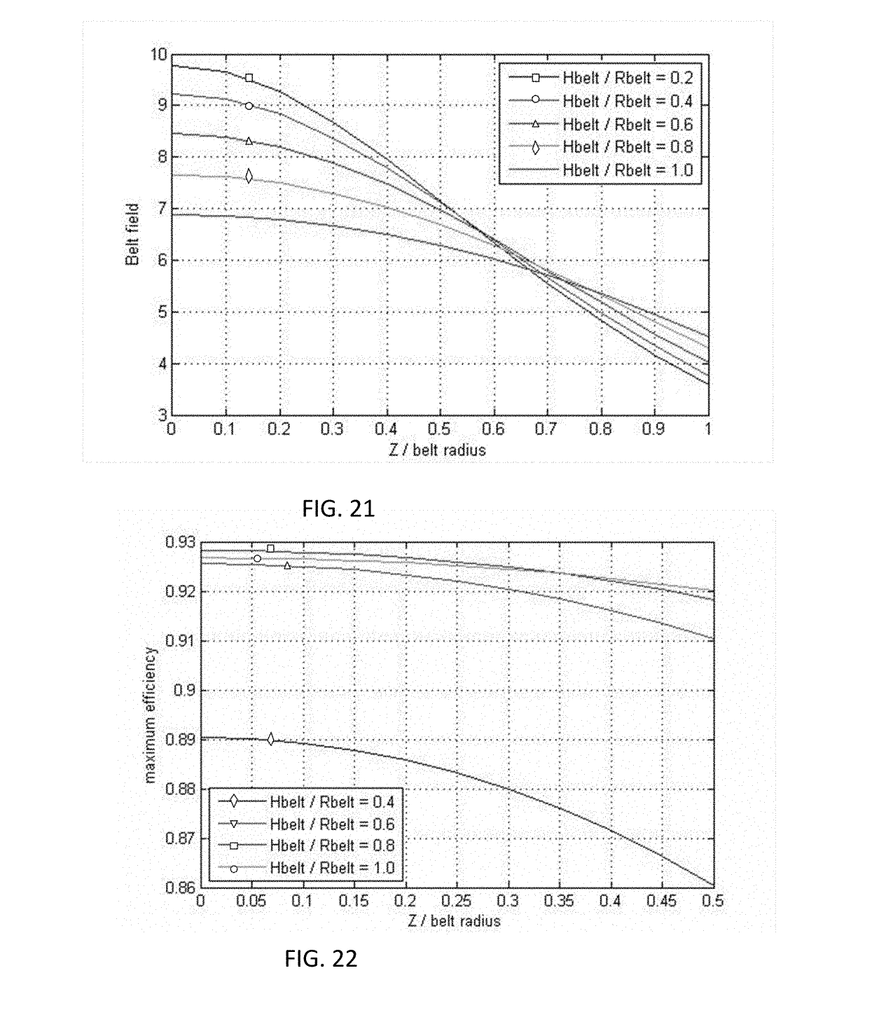

The height H1 of the transmitter coil can be designed to minimize proximity effect. Ideal heights H1 for minimizing proximity effects range from about 3 cm to 20 cm. FIG. 21 illustrates the effect that the height of a transmitter coil has on robustness of power transfer if the proximity effects are ignored. FIG. 22 illustrates the effect that the height of a transmitter coil has when the proximity effects are considered.

In certain embodiments, the height H1 is 0.4-1.5 times the size of the radius (the radius being half of the diameter D). That is, a ratio between the height H1 of the transmitter coil and the radius (D1/2) of the transmitter coil is 0.4-1.5. For example, if transmitter coil has a radius of 15 cm, the ideal height H1 ranges from 6-22.5 cm. In some embodiments, the ratio between the height H1 and the radius (D1/2) is about 0.6, 0.8, or 1. Preferably, the ratio is in the range of about 0.8-1. In certain embodiments, the height H1 for optimum efficiency is 12 cm.

According to certain embodiments, the transmitter diameter D is substantially larger than the distance Z between the center of the transmitter and receiver coils. This configuration reduces the effect of dynamic changes in distance Z during coplanar wireless energy transfer (e.g. due to movement of the transmitter compare to the receiver within the body). As a result, power transfer is more reliable and continuous despite the change in Z. The combination of a) transmitter diameter D larger than the distance Z with b) a height H1 to radius (D/2) ratio in the range of 0.8-1 further improves energy transfer.

The influence of the number of turns N1 on power transmission depends on the type of wire used and the quality factor of the coil. Using litz wires with 100-600 strands/36-48 AWG, the number of turns N1 of the transmitter coil may range from 6 to 35, preferably 20. In addition, the number of turns N1 may be arranged in 1-3 layers. FIG. 26 depicts a transmitter coil having 16 wire turns arranged in two layers (e.g. 8 turns per layer).

In certain embodiments, a capacitor of the transmitter should have an ESR that is less 5× the coil's active resistance and preferably 1/10 of the Coils resistance. Preferred wires for the transmitter are litz wires with 100-600 strands/36-48 AWG.

B. Receiver Geometry and Design

The following are preferable design and geometry details of the receiver.

According to certain embodiments, the diameter d of the receiver coil, as shown in FIG. 25, can be sized to fit to an anatomic location within the patient. For a receiver coil located in the pericardium, the diameter d may range from, for example, about 7 cm (children pericardium) to about 20 cm (adult pericardium and one pleural cavity).

The ideal number of turns N2 of the receiver coil may range from 5 turns to 50 turns. The turns may be arranged in one to three layers (similar to the transmitter coil depicted in FIG. 26). Preferably, the receiver coil includes 20 turns in one layer.

Like the transmitter coil, the receiver coil can be designed to overcome the proximity effect. There are two different receiver coil designs that can be used to overcome the proximity effect. These designs can also be applied to the transmitter coil. The first design is a connected ring coil and the second design is a separated ring coil. Both designs may include a covering around the inductive wires. The covering is preferably biocompatible and provides insulation. Suitable materials include polymers, such as silicone (e.g. NiSil Med 4735 or Med 421), or epoxy materials. Ideally, the receiver coil is able to collect power for the implant, while maintaining enough flexibility to rest within the diaphragm area.

A connected ring coil design is one in which the coil's wire loops or turns are united such that the distance (i.e. pitch) between each turn is substantially constant. In certain embodiments, a single covering layer connects the coil's turns into a single connected structure. Connected ring coils have some flexibility, but the basic structure of the ring coil is maintained with enough rigidity to ensure that the distance (i.e. pitch) between each turn is substantially constant. Having a uniform minimum distance is best for reducing proximity effect. Pitch is the distance between the center of one turn and the next. The structure of the connected ring coil avoids/minimizes proximity effect of electromagnetism. The flexibility of the connected ring coil can be altered to suit, for example, its intended implantation area. The flexibility depends on the materials chosen for the wire and covering—varying based on those materials' parameters for elongation, tensile, durometer, hardness. In one embodiment, the structure of the connected ring coil has a height of about 1 cm to about 3 cm and a pitch of about at least 0.1 inch. In other embodiments, the pitch is at least 0.5 inches. In some embodiments, the pitch between turns is chosen to be substantially equal to the diameter of wire, which acts to further minimize proximity effect. Connected ring coils of the invention ideally have a narrow, small range of resonance frequency, making it easier to gauge and adapt to the resonance frequency. This narrow resonance frequency characteristic simplifies the calibration and control of the system.

A separated ring coil design includes turns (i.e. rings) that are separated to allow variable pitch and other movement between the turns. This provides more flexibility to the receiver geometry. In order to allow variable pitch and other movement, the turns of the coil are not fully connected to each other such that the rings can move away from each other to a desired extent. For example, only a portion of the turns is coupled so there is more movement between the rings. In other words, a separated ring coil is a coil that has one or more connecting points between turns (or rings), which act to provide flexibility in one or more directions, while preventing over-expansion and over-compression. The movement can be in the pitch direction (e.g. movement between turns) or can be in the lateral direction (upward or downward movement of side by side turns). The flexibility of the separated ring coil can be altered to suit, for example, its intended implantation area. The flexibility depends on the materials chosen for the wire and covering—varying based on those materials' parameters for elongation, tensile, durometer, hardness.

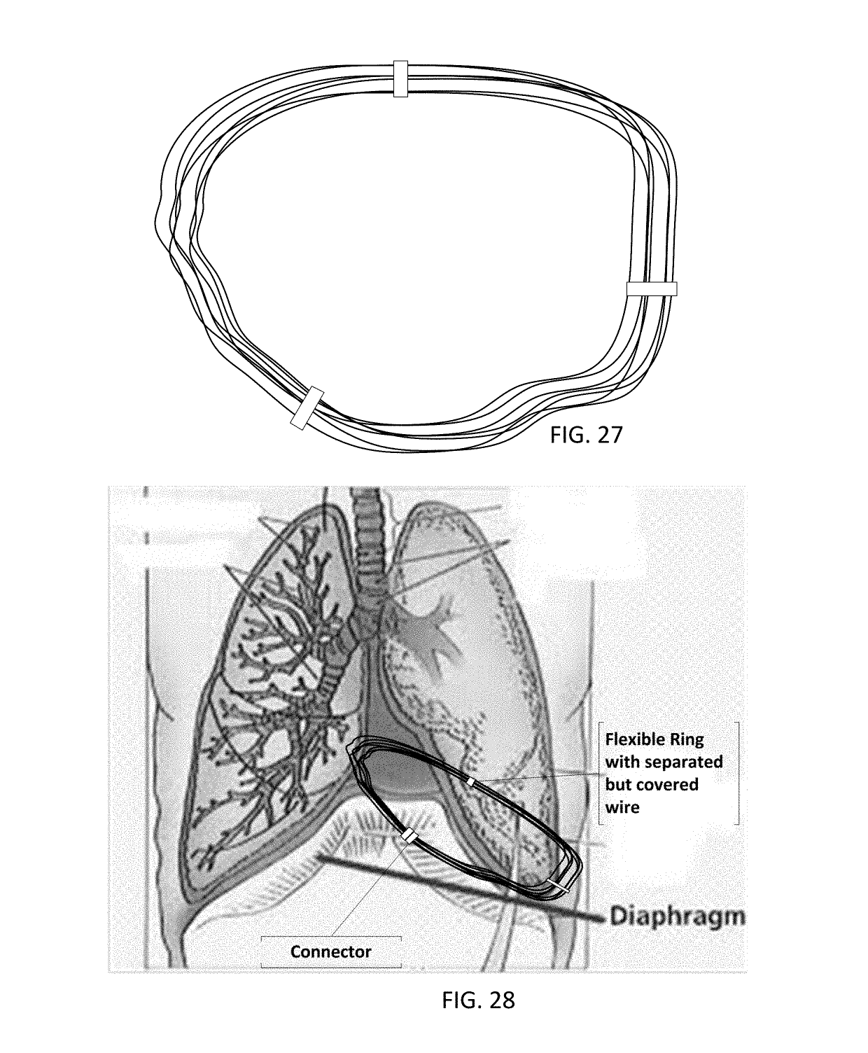

The separated ring coil may also have a covering for insulation, but the covering does not form a single connected structure. Instead, the covering may only partially connect the rings together at one or more points. In certain embodiments, the covering does not connect the rings together, but one or more separate connectors are placed on the coil to connect the rings together (such as the connectors shown in FIGS. 27 and 28). The benefit of the connectors is that a doctor can place them on or manipulate their positions on the ring coil during implantation of the transmitter within the body. This makes it easier to implant the transmitter within the body (such as in the pericardium, pleural cavity, or both). FIG. 28 depicts a separated ring coil disposed within a pleural cavity.

While the variable pitch and added movement of separated coils allows for easier implantation, the variable pitch may result in proximity effect if the wires are too close to each other. In order to prevent proximity effect, the invention provides for covering the wire of the separated ring coil with a covering material of a certain thickness to provide a minimal pitch distance between rings. For example, the covering of the ring coil may be 0.05 inch thick, such that the pitch between any two rings touching each other is at least 0.1 inches. The second receiver coil design for reducing pitch is use of separate but covered flexible wires for the receiver coil. In other embodiments, separated but covered wires are used for the receiver coil.

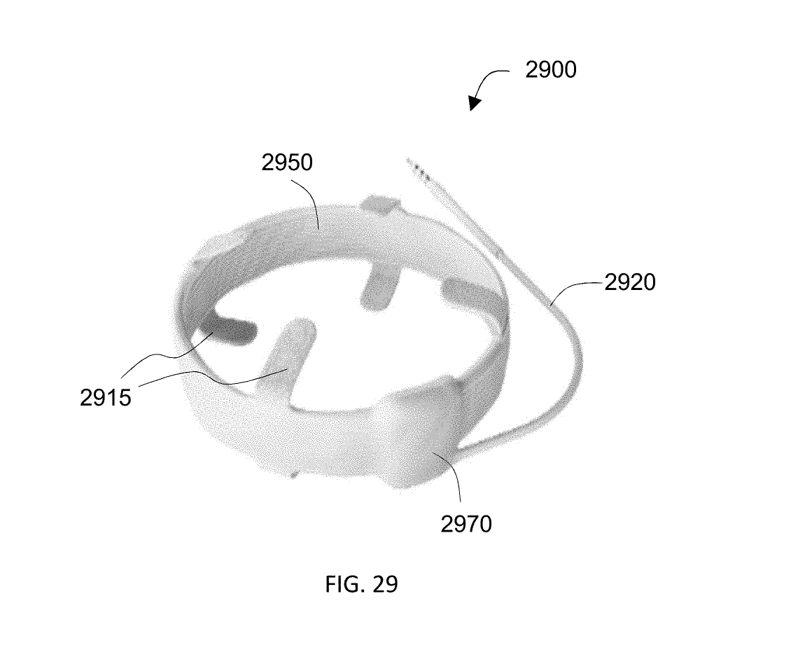

FIG. 29 shows an embodiment of an implantable receiver coil 2900. The implantable receiver coil 2900 includes several tabs 2915 to help secure it to surrounding tissue so that it does not migrate within the body once implanted. A coiled wire is encased in an insulating cover material 2950, which may be a polymer, silicone, or other biocompatible material. Extending from the ring is an electrical lead 2920, which connects to the wire to an implanted medical device (not shown). The electrical lead 2920 extends from the electronics housing 2970, which contains the electrical components which may include a capacitor, a FET switch, a comparator circuit, and other elements of the electrical circuit other than the coil itself. The implantable receiver coil 2900 may have a height of between about 0.1 cm and about 3 cm and a diameter of between about 1 cm and 20 cm, depending on the particular purpose and the size of the area of implantation.

As indicated above, and in FIGS. 1, 2, and 9-11, the implantable receiver coil can be implanted within the abdomen of a patient, such as inside the ribcage and within the chest cavity. A preferred location is the pleural cavity, which is the thin fluid-filled space between the outer (parietal) pleura and the inner (visceral) pleura of each lung. The outer pleura is attached to the chest wall and the inner pleura covers the lungs. Under normal conditions, the pleurae adhere to each other, making the pleural cavity a secure location for implanting a device. As shown in FIG. 10, an implantable receiver coil can be placed in the pleural cavity, beneath the lung and above the diaphragm. As shown in FIG. 10, multiple coils can be placed in the body, such as in the pleural cavity of each lung. That provides a type of redundancy where one coil could potentially act as a backup for the other, or they could alternate between being connected and disconnected so that one coil is connected at all times. As shown in FIGS. 9, and 11, other implantation areas are possible as well, such as the pericardium (FIG. 9) and the aorta (FIG. 11). Multiple coils could be implanted among the various locations, providing redundancy for the power system. The implantable receiver coils can be implanted in one pleural cavity or both, depending on the particular needs of the patient.

As previously discussed, the implantable receiver coil can be implanted elsewhere within the body as well. For example, it can be placed within an extremity such as an arm or leg, or even inside the head or neck. The only limitation on the placement of the implantable receiver coil is that it should be located such that it can be surrounded by an external transmission coil, as described above.

In addition to implanting multiple coils in different parts of the body, another solution for providing redundancy to the system is to implant a single implantable receiver coil that contains multiple separate coils that are configured to operate independently from each other. Although the implantable component of the disclosed coplanar energy transfer system has been referred to throughout as a coil, it should be understood that the implantable device may include multiple individual coiled wires that form circuits that are independent from each other, each of which may function as a receiver coil on its own or in combination with the one or more other coiled wires. With multiple independently functioning receiver wires, the implantable coil can operate as its own backup; if one wire should fail, the unit can continue functioning with another functioning wire. For clarity, in the following discussion, the receiver component of the energy transfer system (which has generally been referred to as an “implantable receiver coil”) will be referred to as a “receiver unit,” and each independently operable wire will be referred to as a “receiver coil.” The receiver unit includes the multiple coils, the electronics, and the covering material.

As explained above, powering an implanted device using wireless coplanar energy transfer avoids the need for transcutaneous wires that must run through a port that is susceptible to infection or other complications. Instead, a receiver unit can be implanted within the body of a patient, where it maintains an electrical connection with an implanted medical device such as a VAD. The receiver unit acts as a receiver antenna for power transmitted by the external transmitter belt. Coplanar energy transfer thus allows an implanted medical device to function for long periods of time with limited complication for the patient. However, by removing the hardline between the implanted medical device and an external power source, wireless systems have the added risk that a failure of the energy transfer system would endanger the patient. If the implanted receiver fails, for example, the patient may require emergency surgery to get the implanted medical device back online. In some situations, depending on the type of implant and the length of time the patient can survive without the implant working, replacing a faulty implanted receiver device may be impractical or impossible.

The robustness of the energy transfer system therefore requires the receiver unit to include a backup mechanism, which allows the receiver to continue receiving power even in the event of a failure. Such a backup mechanism would allow a malfunctioning receiver unit to continue operating. The backup mechanism could be configured to power the implant indefinitely (i.e., just as the main coil) or it could be configured to provide energy for at least long enough for the patient to seek further medical help and have the entire unit replaced.

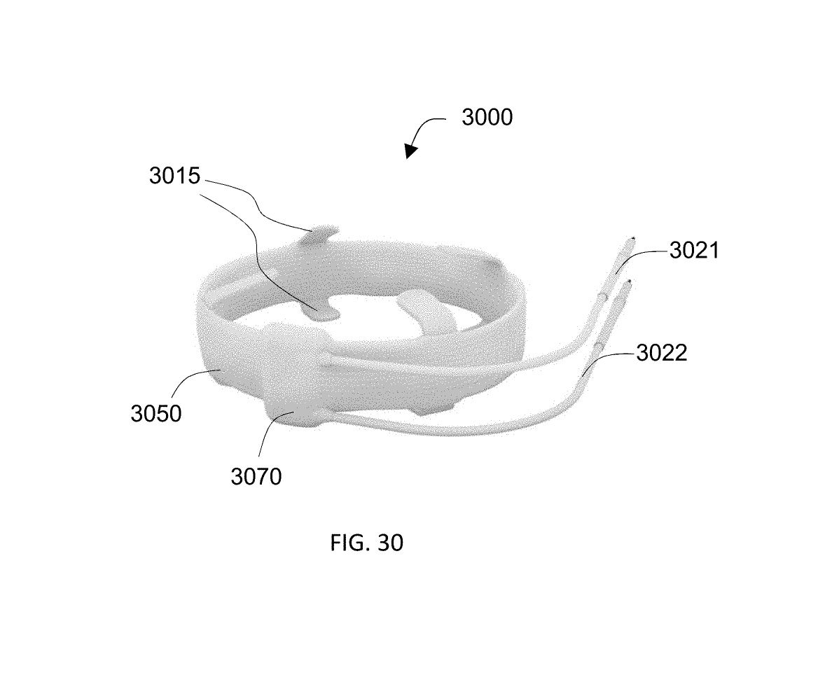

Including multiple coils together in a single implant provides the safety benefits of having a backup or redundant receiver unit, but without the complexity of separately implanting two or more units. The receiver units described below can include multiple receiver coils. For example, it may include two, three, four, five, ten, or more coils. In the embodiments shown in FIGS. 30-32, the receiver unit has two coils.

In some embodiments, a single implantable receiver unit includes multiple receiver coils configured to operate both individually as single receivers and cooperatively as a dual receiver. The unit thus operates when both receiver coils are functional and also when one of the coils fails. In some embodiments, the multi-coil receiver unit is configured so that each coil is only operational when the other is not. The two coils can be switched on and off by controller software.

A problem that arises from having two receiver coils together in a receiver unit is that they must be tuned to be able to work together or a single unit when the other is off and/or has failed. The receiver coil includes an inductor (the coiled wire) and a capacitor to form an LC circuit which is tuned to the desired operation frequency. When the two coils are in proximity to each other they have some coupling between them. The presently disclosed receiver coils can be tuned to reduce or eliminate the mutual effect that occurs between the multiple coils operating in the same field flux environment. Without such tuning, the capacitor on one coil is sensed on the other coil. A receiver unit that has two coils in close proximity has a high coupling factor, such that it would act as one coil. The capacitance needed for achieving resonance would be about half of that required for each individual coil since they would be calculated as two parallel capacitors. A problem would arise if one coil fails or gets disconnected because the capacitor may or may not be disconnected from the cycle and the resonance structure may fail.

The mutual effect problem can be overcome by operating each coil separately, rather than simultaneously. The capacitor of each coil is disconnected when that coil is not operating. It is connected only when power is being harvested by that coil. The disconnected coil does not interfere with the connected coil since there is no current running through it that would interfere with the other active cycle.

Each redundant receiver coil may be substantially similar to the coils that have been described throughout the present disclosure, such as those shown in FIGS. 1-3, 9-11, and 25-28.

FIG. 30 shows an embodiment of a receiver unit 3000 where the two receiver coils are housed within an insulating cover 3050, which may be plastic or some other biocompatible material. The receiver unit may include a plurality of tabs 3015 to help secure it to surrounding tissue so that it does not migrate within the body once implanted. The receiver unit 3000 has two electrical leads 3021 and 3022 extending from the cover, each of which connects to the implanted medical device such as a VAD (not shown). The electrical leads extend from the electronics housing 3070, which contains the electrical components which may include a capacitor, a FET switch, a comparator circuit, and other elements of the electrical circuit other than the coils themselves. In the example shown, leads 3021 and 3022 connect to the first and second receiver coils (obscured from view by the surrounding cover 3050).

The two coils housed within the cover can be configured in various ways. The coils may have either the connected ring design or the separated ring designs described above and shown in FIGS. 25-28. Both designs may include a covering around the inductive wires as seen in FIG. 30. The covering is preferably biocompatible and provides insulation. Suitable materials include polymers, such as silicone (e.g. NiSil Med 4735 or Med 421), or epoxy materials. Ideally, the receiver coil is able to collect power for the implant, while maintaining enough flexibility to conform to the anatomy of the location of implantation.

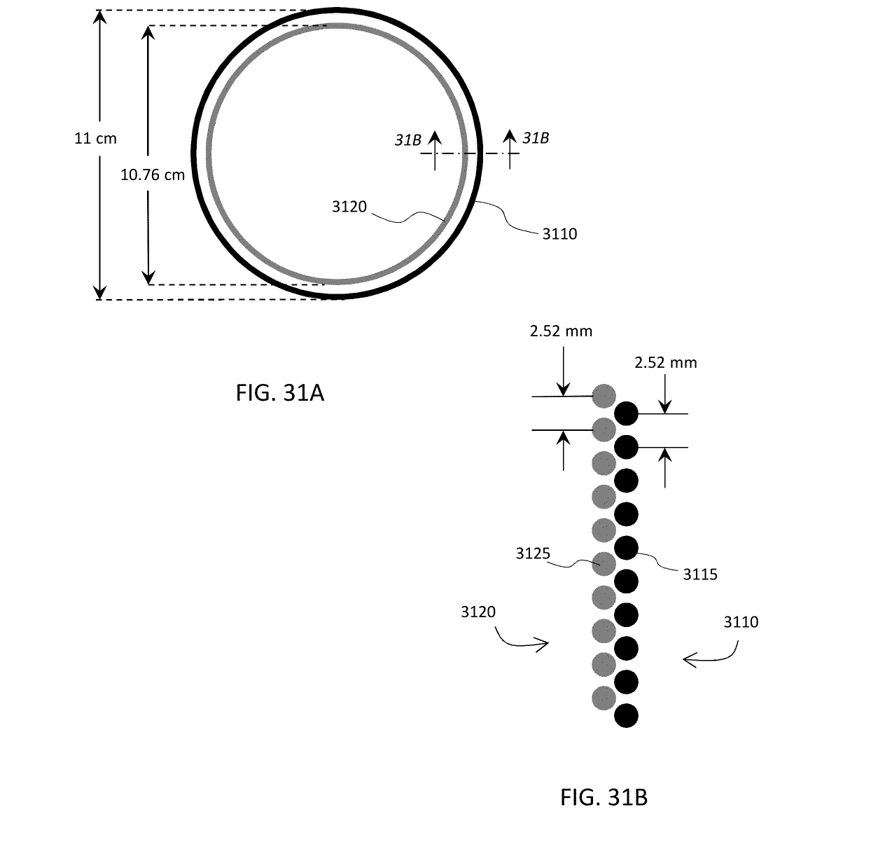

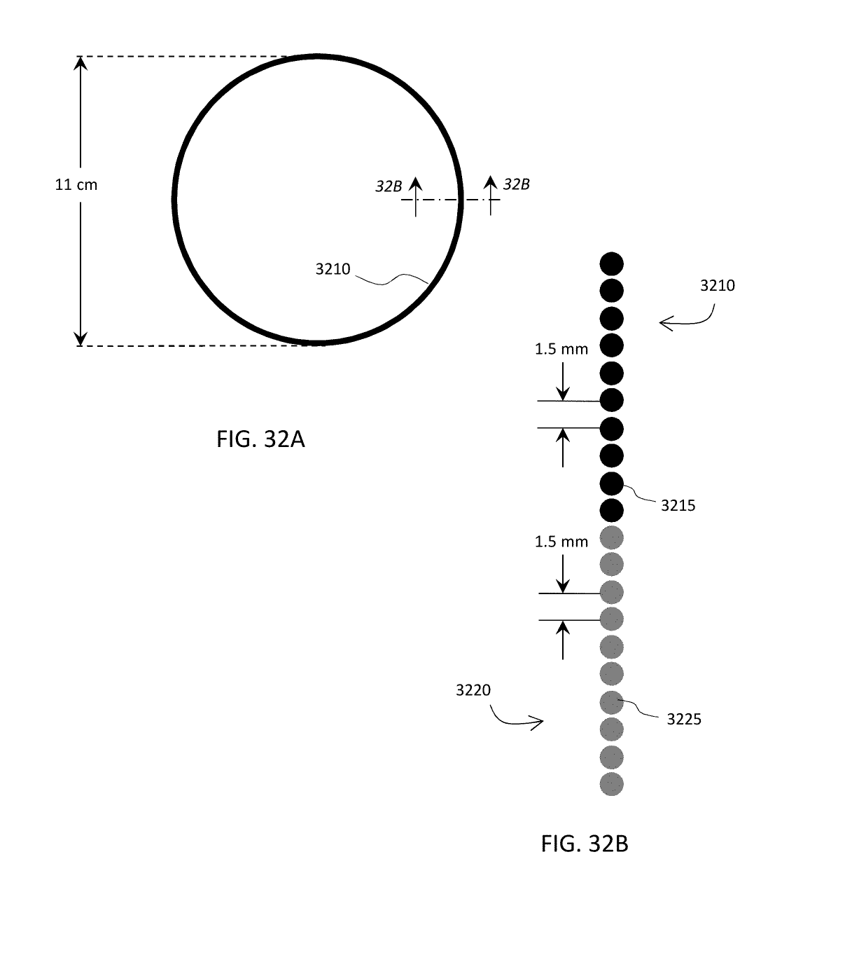

The arrangement of the first and second receiver coil wires within the cover material may assume various geometries. FIGS. 31A-B show one possible configuration. FIG. 31A is a top view of two coils, where a first coil 3110 has a diameter of 11 cm and the second coil 3120 has a diameter of 10.76 cm. The second coil 3120 fits inside the first coil 3110. FIG. 31B shows a cross-section of the coils. The first coil 3110 has ten turns 3115 and the second coil 3120 also has ten turns 3125. In other embodiments, the coils may have different numbers of coils from each other. As shown, the turns 3125 of the second coil 3120 are configured to nest within the spaces between the turns 3115 of coil 3110, so that they overlap to some extent, albeit without touching. The pitch of the wires is 2.52 mm for both coils, which is greater than the thickness or gauge of the wire itself, so each turn of the wire does not touch the neighboring turns. In FIGS. 31A-B, as well as FIGS. 32A-B and 33A-B, the different coils are illustrated as light and dark to help distinguish them from each other.

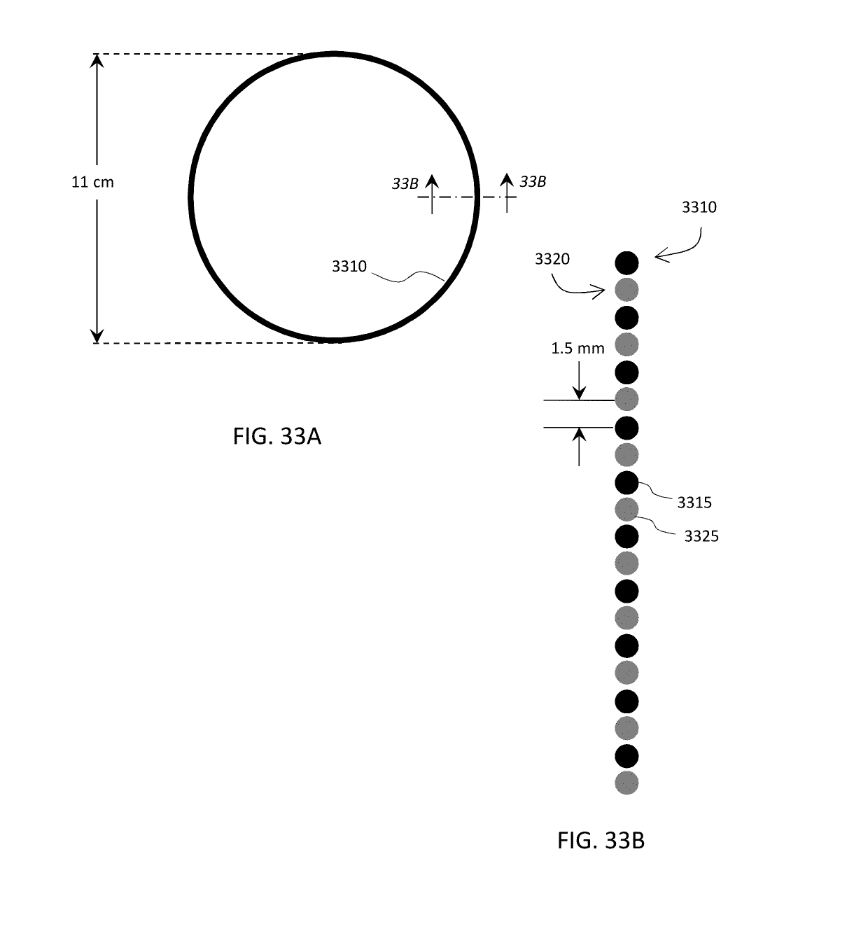

FIGS. 32A-B show another configuration of two receiver coils 3210 and 3220. Here the two coils each have a diameter of 11 cm and are stacked directly on top of each other, so that only one of them is visible from the top view shown in FIG. 32A. The cross-section of FIG. 32B shows that, like the embodiment of FIGS. 31A-B, each coil has ten turns 3215 and 3225 of wire, but because they are stacked on top of each other the overall height of this embodiment is greater than in the embodiment where the coils are nested with each other. By comparing FIGS. 31B and 32B it can be seen that the nested embodiment has a lower profile than the stacked embodiment. The pitch between the turns of coil 3210 and coil 3220 is 1.5 mm. The pitch can be varied as necessary. Likewise, in FIGS. 33A-B, a receiver unit has a first receiver coil 3310 and second receiver coil 3320, each having the same diameter (11 cm). But while in FIGS. 32A-B the receiver coils 3210 and 3220 are stacked on top of each other, in FIGS. 33A-B, the two receiver coils 3310 and 3320 interwoven with each other, as shown in the cross-section of FIG. 33B. The twenty wire turns alternate between the first coil's turns 3315 and the second coil's turns 3325. It should be understood that the three embodiments depicted in FIGS. 31A-33B are exemplary only, and are not meant to be limiting. The different possible configurations may be more or less desirable based on the electrical properties and interactions of the coils. A person of ordinary skill in the art would recognize other similar configurations in which to arrange the two coils.

In certain embodiments, the two receiver coils are interleaved with each other. Each coil operates only when the other is disconnected. In FIGS. 31A-B, for example, the two receiver coils are interleaved but slightly offset from each other, whereas in FIGS. 33A-B the two receiver coils are fully interleaved with each other. As depicted, the two coils are identical in thickness or gauge, having an equal number of strands making up the wires. In such embodiments, the coils can be switched on and off periodically to achieve better heat dissipation.

The coils can be switched, for example, at intervals of a few seconds. In other embodiments, the two coils may be of different sizes. The coils can have a master/slave configuration where one coil is a Litz wire with many strands (for example, 200 or more), which allows high efficiency, and the other coil is a smaller size Litz wire with fewer strands (for example, 50 or fewer), which provides minimum operation efficiency. The smaller coil is intended for redundancy if the larger coil should fail.

Although specific diameters are shown in FIGS. 31A-33B, it should be understood that the diameter of the receiver coil can be sized to fit the location where it will be implanted. The diameter can be 1 cm or less, or it can be for example, 5, 10, or 20 cm. The receiver coil may have a plurality of turns, ranging from about 5 to about 50 turns. In some embodiments, each coil has 10 turns. The turns are generally arranged one on top of the next, so that each coil roughly forms a spring-shape. In some embodiments, the coils may form layers that fit inside one another, or one coil may fit within the other coil so that together they form a smaller profile receiver unit.

Like the coils described above, the redundant receiver coils can be designed to have heights and pitches that help to overcome the proximity effect between turns of the coils. Generally, the receiver coil's turns have a substantially constant pitch between them. The insulating cover holds the turns in place to maintain the pitch. Since the receiver coil has some degree of flexibility, the pitch may vary slightly along the length of the wire, but it is rigid enough to maintain a generally constant pitch, with a variation of preferably no more than about 0.01 inches. The wires may include a covering material of a certain thickness to provide a minimal pitch distance between rings. For example, the covering of the ring coil may be 0.05 inch thick, such that the pitch between any two adjacent rings is at least 0.1 inches. The receiver unit may have a height of about 1 cm to about 3 cm. The coils may have a pitch of between 0.05 inches and 0.5 inches. The pitch may approximately equal to the diameter of wire. In some embodiments (shown in FIGS. 33A-B) the pitch refers to the distance between the centers of adjacent turns, which belong to separate interleaved coils.

In an embodiment, the two receiver coils are each configured to operate one at a time, while the other is in a disconnect phase. In another embodiment, the two receiver coils are configured to operate simultaneously, and either individual coil can continue operating if the other should fail.

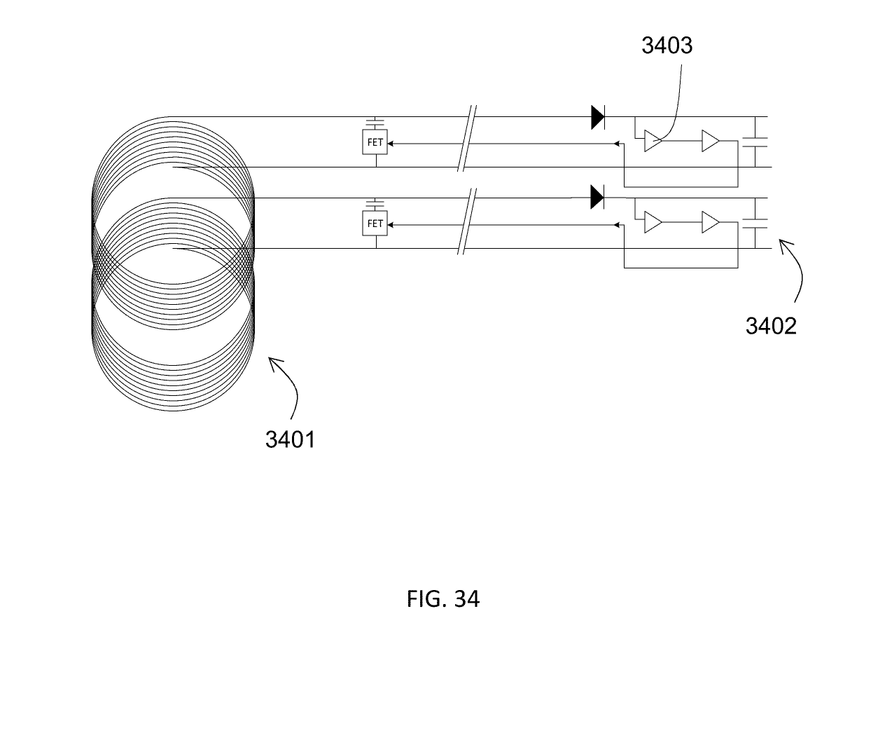

FIG. 34 depicts an example of the circuit configuration for two receiver coils operating in parallel. Each receiver coil forms an LC circuit with an inductor coil 3401, a capacitor 3402 and a FET switch. The switch turns one of the coils off while the other operates. Without the switch, the circuits would interfere with each other or couple in a destructive way even though they are not galvanically connected. As shown, each circuit includes a comparator 3403, which allows the system to determine if the voltage is higher than desired, thereby causing the FET switch to turn off. Alternatively, the two circuits can alternate between on and off by controller software.

The two receiver coils do not need to be symmetrical. The redundant coil may have a much lower number of strands as compared to the main coil. For example, the main coil may have 200 strands and the redundant coil only 40 strands. As a result, the main coil would have a resistance of approximately 0.06 ohms (compared to 0.33 ohms for the redundant coil). The redundant coil would be less efficient, but it would not affect the overall robustness of the system.

The two receiver coils can function cooperatively in various ways. In one embodiment, the first receiver coil maintains an electrical connection to the implanted medical device, and the second receiver coil is disconnected from the implanted medical device. The second receiver coil functions as a reserve in the event that the first receiver coil fails. In another embodiment, the first and second receiver coils alternate between being on and off, with one or the other providing power at any given time and the other being disconnected. The switching can be controlled by software.

Various modifications may be made to the embodiments disclosed herein. The disclosed embodiments and details should not be construed as limiting but instead as illustrative of some embodiments and of the principles of the invention.

What is claimed is:

1. A system for wirelessly powering an implanted medical device, comprising:
a transmitter inductive coil configured to be disposed externally around a part of a body of a patient within which the medical device is implanted such that the transmitter inductive coil surrounds that part of the body, wherein the transmitter inductive coil is configured to inductively transfer electromagnetic power into the part of the body; and
a receiver inductive coil configured to be implanted within the part of the patient's body, the receiver inductive coil comprising a first wire and a second wire, wherein each wire is electrically connected to the medical device and is operable to wirelessly receive the inductively-transferred electromagnetic power from the transmitter inductive coil and provide the received power to the medical device.

2. The system of claim 1, wherein the first wire and the second wire are operable independently of one another.

3. The system of claim 1, wherein the first wire and the second wire are configured to operate simultaneously.

4. The system of claim 1, wherein each wire is configured to carry alternating currents.

5. The system of claim 1, wherein the first wire is disposed concentrically around the second wire.

6. The system of claim 1, wherein each wire comprises a plurality of turns.

7. The system of claim 6, wherein the turns of the first wire are interwoven with the turns of the second wire.

8. The system of claim 6, wherein the wires have a pitch of at least 0.05 inches between each turn.

9. The system of claim 8, wherein each of the turns is connected such that the pitch is substantially constant.

10. The system of claim 8, wherein the turns are coupled such that the pitch is variable, and the receiver inductive coil comprises a covering such that the pitch between any two wire turns is 0.05 inches or more.

11. The system of claim 1, wherein one or both of the first and second wires comprises 20 to 600 strands.

12. The system of claim 11, wherein the first wire has a greater number of strands than the second wire.

13. The system of claim 1, wherein the receiver inductive coil is associated with an AC-to-DC rectification circuit that comprises a single diode.

14. The system of claim 1, wherein the medical device is a ventricular assist device (VAD).

15. A receiver inductive coil associated with a medical device and configured to be implanted within a part of a body of a patient, the receiver inductive coil comprising a first wire and a second wire, separate from the first wire, each wire configured to wirelessly receive inductively-transferred electromagnetic power from a transmitter inductive coil external to the part of the patient's body and provide the received power to the medical device.

16. The receiver inductive coil of claim 15, wherein each wire comprises 20 to 600 strands.

17. The receiver inductive coil of claim 16, wherein the first wire has a greater number of strands than the second wire.

18. The receiver inductive coil of claim 16, wherein each wire has a pitch of at least 0.05 inches between each turn.

19. The receiver inductive coil of claim 15, wherein the turns of the first wire are interwoven with the turns of the second wire.

20. The receiver inductive coil of claim 15, wherein the first wire is disposed concentrically around the second wire.

21. The receiver inductive coil of claim 15, wherein the first wire and the second wire are operable independently of one another.

22. The receiver inductive coil of claim 15, wherein the first wire and the second wire are configured to operate simultaneously.

23. The receiver inductive coil of claim 15, wherein each wire comprises 100 to 600 strands with a gauge ranging from 36 AWG to 48 AWG.

24. The receiver inductive coil of claim 15, wherein each wire is configured to carry alternating currents.

25. The receiver inductive coil of claim 16, wherein the turns of each wire are connected such that the pitch is substantially constant.

26. The receiver inductive coil of claim 16, wherein the turns of each wire are coupled such that the pitch is variable, and the receiver inductive coil comprises a covering such that the pitch between any two wire turns is 0.05 inches or more.

27. The receiver inductive coil of claim 15, wherein the receiver inductive coil is associated with an AC-to-DC rectification circuit that
